(19) United States
(12) Patent Application Publication
(10) Pub. No.: US 2024/0216372 A1
(43) Pub. Date: Jul. 4, 2024

(54) THERAPEUTICS FOR KELOIDS AND OTHER FIBROTIC DISORDERS

(71) Applicant: Florida State University Research Foundation, Inc., Tallahassee, FL (US)

(72) Inventors: Akash GUNJAN, Tallahassee, FL (US); Yuna SON, Waltham, MA (US)

(21) Appl. No.: 18/398,315

(22) Filed: Dec. 28, 2023

Related U.S. Application Data
(60) Provisional application No. 63/435,626, filed on Dec. 28, 2022.

(51) Int. Cl. A61K 31/506; A61K 31/136; A61K 31/167; A61K 31/405; A61K 31/4418; A61K 31/496; A61K 45/06; A61P 17/02

(52) U.S. Cl. CPC A61K 31/506; A61K 31/136; A61K 31/167; A61K 31/405; A61K 31/4418; A61K 31/496; A61K 45/06; A61P 17/02

(57) ABSTRACT

This disclosure relates to pharmaceutical compositions comprising one or more therapeutic agents selected from a SERPINE1 serine protease inhibitor, a histone acetyltransferase inhibitor, a histone deacetylase inhibitor, an insulin-like growth factor inhibitor, an anti-hypertensive agent, a topoisomerase II inhibitor, a tyrosine kinase inhibitor, an agent that downregulates growth factors or any combination thereof, and methods of treating fibrotic disorders, including keloid, pulmonary fibrosis, hepatic fibrosis, cardiac fibrosis, renal fibrosis, mediastinal fibrosis, retroperitoneal cavity fibrosis, bone marrow fibrosis, and/or scleroderma using the same. The disclosed methods can be accompanied by application of radiation and/or surgical resection. For treating keloids, the compositions can be formulated topically and do not cause systemic side effects. For treating fibrotic diseases of internal organs, the compositions can be administered systemically or delivered to the affected organs. The compositions can include two or more therapeutic agents that work synergistically, allowing lower doses of each therapeutic agent.

### CROSS REFERENCE TO RELATED APPLICATIONS

This application claims the benefit of U.S. Provisional Application No. 63/435,626 filed on Dec. 28, 2022, which is incorporated herein by reference in its entirety.

### STATEMENT REGARDING FEDERALLY SPONSORED RESEARCH OR DEVELOPMENT

This invention was made with government support under subaward SUB00002344 of grant number UL1TR001427 awarded by the National Center for Advancing Translational Sciences of the National Institutes of Health. The government has certain rights in the invention.

### BACKGROUND

Keloids are disfiguring, painful, and itchy, but benign fibrotic skin tumors or lesions characterized by excessive dermal fibroblast proliferation and collagen deposition. They occur in susceptible individuals due to abnormal wound healing. Keloids are understudied, difficult to treat, and predominantly affect dark-skinned individuals. Their incidence is estimated to be as high as 1 in 6 among African Americans, compared to around 1 in 1000 among white Americans, suggesting that genetic and/or epigenetic factors contribute strongly to keloid disease. However, the molecular mechanisms involved in driving keloid formation in susceptible individuals are unclear.

Currently there are no standardized treatments available for keloids and they have very high recurrence rates upon surgical resection alone. Intralesional steroids are commonly used, albeit with highly variable responses. Furthermore, although superficial radiation therapy is emerging as one of the most effective post-surgical adjuvant therapies to prevent keloid recurrence, it requires specialized equipment and its association with cancer therapy leads to reluctance among both providers and patients for its use in keloid therapy.

Despite advances in medical research, there is still a scarcity of methods and compositions that are effective in the treatment of keloids that do not require specialized equipment and/or trips to a medical facility, as does radiation therapy. An ideal drug composition for the treatment of keloids could be applied topically by or administered intralesionally to subjects in need thereof, would not have systemic side effects, and could work synergistically, allowing lower doses. These needs and other needs are satisfied by the present disclosure.

### SUMMARY

In accordance with the purpose(s) of the present disclosure, as embodied and broadly described herein, the disclosure, in one aspect, relates to pharmaceutical compositions comprising one or more therapeutic agents selected from a SERPINE1 (PAI1) serine protease inhibitor, a histone acetyltransferase (HAT) inhibitor, a histone deacetylase (HDAC) inhibitor, an insulin-like growth factor (IGF) inhibitor, an anti-hypertensive agent, a topoisomerase II inhibitor, a tyrosine kinase inhibitor, an agent that downregulates growth factors or any combination thereof, and methods of treating fibrotic disorders, including keloid, pulmonary fibrosis, hepatic fibrosis, cardiac fibrosis, renal fibrosis, mediastinal fibrosis, retroperitoneal cavity fibrosis, bone marrow fibrosis, scleroderma or systemic sclerosis, and combinations thereof using the same. The disclosed methods can be accompanied by application of radiation and/or surgical resection. For treating dermatological fibrotic diseases like keloids, the compositions can be formulated topically and do not cause systemic side effects. For treating fibrotic diseases of internal organs, the compositions can be administered systemically, or delivered directly to the affected organs by any appropriate means. In another aspect, the compositions include two or more therapeutic agents that work synergistically, allowing lower doses of each therapeutic agent.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows details of workflow used for combining enzymatic DNA methylation sequencing (EMseq), microRNA sequencing (miRNAseq), and RNA sequencing (RNAseq) datasets for a “multiomics” approach to identify the major genes driving keloid pathogenesis. EMseq performed in duplicate revealed 35,935 Differentially Methylated Regions (DMRs) in keloid DNA samples corresponding to 2,524 genes with enhanced DNA methylation and 4,546 genes with reduced DNA methylation. The EMseq data was correlated with the miRNAseq data on the targets of the Differentially Expressed miRNAs (DEMs) present in keloids, as well as the Differentially Expressed Genes (DEGs) identified by RNAseq. For example, since DNA methylation leads to gene silencing, a highly methylated gene from the EMseq dataset corresponding to its downregulated transcript levels in keloid samples in the RNAseq data, or vice versa, would be a positive correlation and generate more confidence in that candidate gene playing a potentially important role in keloid pathology. Similarly, a miRNA overexpressed in keloids with its target genes being downregulated in the RNAseq data, or vice versa, would also boost confidence that these genes are likely to be contributing to the keloid phenotype.

FIGS. 2A-2D show identification of actionable genes and pathways driving keloid pathogenesis using our multiomics approach. FIG. 2A: MA plot of RNA sequencing (RNAseq) performed in triplicate on 4 normal Human Dermal Fibroblasts (HDFs) and 6 keloid dermal fibroblast samples from patients (P) shows the upregulation of 1,418 and downregulation of 2,073 genes for a total of 3,491 Differentially Expressed Genes (DEGs) in keloid samples. Gray dots denote higher mean expression levels and black dots denote lower mean expression levels (log 2FC: log 2 fold change) in keloids dermal fibroblasts compared to the normal HDFs. FIG. 2B: Heatmap of the top 20 up- and downregulated genes identified in the RNAseq data are shown, several of which have been implicated in keloid pathogenesis previously, while others have not been studied so far in the context of keloids. FIG. 2C: Pie chart depicting the results of the integrated data from 3481 DEGs from RNAseq, 7070 DMRs from EMseq and the DEMs from miRNAseq data resulted in a relatively short list of 181 genes (132 genes that were upregulated 49 genes that were downregulated) that would be our high confidence candidate genes involved in keloid pathology. Consistent with this notion, two-thirds of these genes function in pathways that are involved in wound healing, extracellular matrix organization and regulation of endothelial cell proliferation—these pathways have been previously implicated in keloid pathogenesis as well as in other fibrotic diseases. Genes highlighted in yellow are known to contribute to fibrotic diseases or keloids via multiple pathways. This clearly validates our integrated multiomics approach for winnowing down large candidate gene lists from individual genomic assays to arrive at the most relevant genes contributing to the biological process under study. Several of the genes identified here have been evaluated as potential drug targets in the rest of figures shown here. Pathways highlighted in yellow potentially contribute to the regulation of blood pressure. High blood pressure, or hypertension, has been previously implicated in keloid pathology, although no molecular data in support of this idea was available previously. FIG. 2D: The FDA approved calcium channel blocking antihypertensive drug Verapamil inhibits the proliferation of keloid fibroblasts. Data from keloid dermal fibroblasts from 4 patients (KPA, P5, P14 and P17) are shown here.

FIGS. 3A-3D show chromeceptin, an inhibitor of Insulin-like Growth Factor 2 (IGF2) signaling, blocks keloid fibroblast proliferation. FIG. 3A: Scheme depicting the negative translational regulation of IGF2 mRNA by the binding of Insulin-like Growth Factor 2 mRNA Binding Protein 1 (IGF2BP1). Chromeceptin is a pre-clinical inhibitor of IGF2 signaling that is effective in the low nanomolar range. The expression of IGF2 and IGF2BP1 are anti-correlated as shown by the data in next two figures. FIG. 3B: Quantitative RT-PCR based validation of the overexpression of IGF2, a signaling protein that stimulates growth and development, in keloid fibroblasts. Data was obtained from 3 normal primary human dermal fibroblasts (HDFs) and 3 keloid dermal fibroblasts. Relative expression was normalized to the levels of HPRT gene expression. Error bars represent standard deviation of the mean (mean±SD) obtained from three experiments. Significant differences are indicated by the asterisk (*P<0.05; ** P<0.005). FIG. 3C: Quantitative RT-PCR confirming the reduced expression of Insulin-like IGF2BP1, a negative regulator of IGF2 translation, in keloid fibroblasts. Experiment was performed as described above for FIG. 3B. FIG. 3D: Keloid dermal fibroblasts are sensitive to Chromeceptin. Data from keloid dermal fibroblasts from 6 patient (P) tissue samples numbered PA, P5, P14, P16, P17 and P25 are shown here.

FIG. 4 shows physiological pathways regulated by SERPINE1 (Serine Proteinase Inhibitor, Family E, Member 1). SERPINE1, also known as Plasminogen Activator Inhibitor 1 (PIA1), is a protease inhibitor that regulates multiple pathways by blocking fibrin and extracellular matrix degradation, thereby leading higher levels of collagen deposition. Legend: urokinase-type plasminogen activator (uPA); tissue-type plasminogen activator (tPA); matrix metalloproteinase (MMP); hepatocyte growth factor (HGF); plasminogen activator inhibitor 1 (PAI1).

FIGS. 5A-5C show Tiplaxtinin, a SERPINE1 inhibitor, preferentially blocks keloid fibroblast proliferation over normal HDF proliferation. FIG. 5A: Quantitative RT-PCR to confirm the overexpression of SERPINE1 in keloid fibroblasts compared to HDFs. FIG. 5B: Normal primary HDFs are not significantly sensitive to Tiplaxtinin, a pre-clinical inhibitor of SERPINE1. FIG. 5C: Keloid dermal fibroblasts are sensitive to Tiplaxtinin in the low micromolar range.

FIGS. 6A-6C show the FDA approved hair loss treatment Minoxidil inhibits Procollagen-Lysine, 2-Oxoglutarate 5-Dioxygenase 2 (PLOD2) and blocks keloid fibroblast proliferation FIG. 6A: Scheme showing the known roles of PLOD2 in regulating collagen maturation, thereby impacting the Extra Cellular Matrix (ECM), which in turn affects metastasis of cancer cells, and presumably keloid formation as well. PLOD2 catalyzes the hydroxylation of lysyl residues in collagen which is critical for the stability of intermolecular crosslinks in mature collagen. FIG. 6B: Quantitative RT-PCR to confirm the overexpression of PLOD2 in keloid fibroblasts compared to HDFs. FIG. 6C: Keloid dermal fibroblasts are sensitive to Minoxidil in the low to sub-millimolar range. Note: Minoxidil is FDA-approved for treating hair loss, where it is typically applied as a 5% (˜45 mM) topical solution.

FIGS. 7A-7C show keloid fibroblasts exhibit higher levels of histone H3 lysine 18 acetylation (H3K18ac) and are sensitive to Histone Acetyltransferase (HAT) inhibitors presumably due to their addition to higher levels of H3K18ac for survival and proliferation. FIG. 7A: Indirect immunofluorescence microscopy using H3K18ac specific antibodies reveals the presence of higher levels of this epigenetic mark in keloid fibroblasts (KPA) when compared to normal dermal fibroblasts (HDF PA) in the same patient. FIG. 7B: Quantitation of the data shown above in FIG. 7A. Ac=acetylation. FIG. 7C: Compared to normal HDFs, keloid fibroblasts are much more sensitive to both natural HAT inhibitors like Curcumin (CUR), as well as synthetic pre-clinical HAT inhibitor compounds such as A485. A structurally related dummy compound, A486, did not affect the proliferation of either HDFs or keloid fibroblasts and serves as a good negative control. Legend: histone acetyltransferase (HAT); curcumin (CUR; a natural HAT inhibitor): dummy compound (A486); synthetic HAT inhibitor (A485).

FIG. 8 shows keloid fibroblasts are exquisitely sensitive to multiple FDA approved Histone Deacetylase (HDAC) inhibitors. Since keloid fibroblasts exhibit higher levels of H3K18ac (FIGS. 7A-7B), it is possible that treatment with HDAC inhibitors may enhance their acetylation levels even further, leading to cytotoxicity. We tested this possibility and found that both keloid fibroblasts and normal HDFs show substantial sensitivity to FDA approved HDAC inhibitors. However, since keloids are a dermatological disorder, it would still be possible to apply these drugs topically or deliver them via intralesional injections in a manner that is localized just to the keloids to derive therapeutic benefits. Legend: vorinostat (VOR); belinostat (BEL); panobinostat (PANO); valproic acid (VPA).

FIG. 9 shows synergistic effects of combination drug treatment on keloid fibroblast proliferation using FDA approved and pre-clinical drugs. Combination drug treatment targeting keloid fibroblast proliferation based on the actionable genes and pathways identified through our multiomics approach or basic research, can lead to synergistic inhibition of keloid fibroblast proliferation at lower doses of individual drugs. A proof of principle experiment is shown demonstrating the enhanced efficacy of a combination of two drugs in inhibiting keloid fibroblast proliferation compared to the individual drugs. In principle, multiple drugs targeting different pathways that independently contribute to keloid formation can be used simultaneously to achieve synergistic benefits in keloid therapy, especially when applied topically to avoid systemic effects. Legend: chromoceptin (CHR); minoxidil (MINO); tiplaxtinin (TIP); vorinostat (VOR).

FIG. 10 shows proliferation of keloid fibroblasts is blocked in a synergistic manner upon treatment with a combination of FDA approved anti-inflammatory and antifibrotic drugs. Patient derived KPA keloid fibroblasts were either left untreated or treated with the indicated concentrations of the drugs, either with or without a low 1 Gy dose of radiation, and surviving cells were counted one week later. Error bars represent standard deviation. TA=Triamcinolone Acetonide, and FDA approved inti-inflammatory steroid commonly used in keloid therapy; PIR=Pirfenidone, a FDA approved antifibrotic drug that works by blocking TGF-β activities that drive keloid disease; NIN=Nintedanib, a FDA approved antifibrotic kinase inhibitor that blocks the function of several growth factors important for keloid fibroblast proliferation; IR=Ionizing Radiation, which has been previously shown by us to be effective in blocking keloid fibroblast proliferation in vitro and preventing keloid recurrence in patients following their surgical excision. Note that this assay cannot accurately determine cell survival below ˜20% due to the presence of significant numbers of senescent cells that are still alive, but which are not capable of proliferating or dividing any more. The use of a higher dose of radiation is likely to result in further synergy in the anti-proliferative effects of the drugs used here. The synergistic effects of the drugs are likely due to the fact that they have different targets in the pro-fibrotic pathways and suggest that significantly improved therapeutic benefits can be obtained by combining these drugs rather than using them individually for keloid therapy.

FIGS. 11A-11H show proliferation of keloid fibroblasts is blocked in a synergistic manner upon treatment with multiple combinations of FDA approved drugs along with radiation. The experiment was performed as described in FIG. 10 using the indicated concentrations of drugs, either without radiation (dark bars) or with ionizing radiation (gray bars). Drugs used here are: Mino=Minoxidil, an inhibitor of Procollagen-Lysine, 2-Oxoglutarate 5-Dioxygenase 2 (PLOD2); Mitox=Mitoxantrone, a DNA intercalating Topoisomerase II inhibiting antineoplastic chemotherapeutic; Nin=Nintedanib, an FDA approved antifibrotic kinase inhibitor; Pir=Pirfenidone, an FDA approved antifibrotic drug; TA=Triamcinolone Acetonide, an FDA approved anti-inflammatory steroid; Vera=Verapamil, a calcium channel inhibiting antihypertensive drug; Vor=Vorinostat, a histone deacetylase (HDAC) inhibitor.

FIGS. 12A-12B show the WNT pathway is upregulated in keloid fibroblasts that are sensitive to inhibition of the WNT pathway using repurposed FDA approved drugs. FIG. 12A shows qRT-PCR based analysis to confirm the differential mRNA expression of WNT5A in keloid fibroblasts relative to normal human dermal fibroblasts (HDF). Primary keloid fibroblasts were obtained and processed for qRT-PCR 3 normal fibroblasts and 3 keloid fibroblasts from 6 patients. Relative expression was normalized to the levels of HPRT gene expression. Error bars represent standard deviation of the mean obtained from three experiments. FIG. 12B shows patient derived keloid fibroblasts were either left untreated or treated with the indicated low concentrations of the FDA approved drugs that are also known to inhibit WNT signaling. Higher concentrations of the WNT inhibiting drugs are expected to have stronger effects. CEL=Celecoxib; Salinomycin=SLM; SUL=Sulindac.

FIG. 13 shows lysyl oxidase-like 2 (LOXL2) gene expression is upregulated in keloid fibroblasts. qRT-PCR based analysis to confirm the differential mRNA expression of LOXL2 in keloid fibroblasts relative to normal human dermal fibroblasts (HDF). Primary keloid fibroblasts were obtained and processed for qRT-PCR 3 normal fibroblasts and 3 keloid fibroblasts from 6 patients. Relative expression was normalized to the levels of HPRT gene expression. Error bars represent standard deviation of the mean obtained from three experiments. Significant differences are indicated by the asterisk (*P<0.05).

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DETAILED DESCRIPTION

Disclosed herein is a method for treating or delaying the onset, progression, or relapse of a fibrotic disorder in a subject, the method including at least the step of administering to the subject one or more therapeutic agents comprising a SERPINE1 (PAI1) serine protease inhibitor, a histone acetyltransferase (HAT) inhibitor, a histone deacetylase (HDAC) inhibitor, an insulin-like growth factor (IGF) inhibitor, an anti-hypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that a HAT inhibitor and an HDAC inhibitor are not administered to the subject simultaneously. Without wishing to be bound by theory, HAT inhibitors and HDAC inhibitors target the same pathway in opposing directions and thus may cancel each other's effects if used together. In some aspects, the method can include administering at least two, three, or four of the disclosed therapeutic agents to the subject in need thereof. In an aspect, any drug from a given class can be used interchangeably for the disclosed purposes. In some aspects, the method can include administering at least two anti-hypertensive agents to the subject. In one aspect, the fibrotic disorder can be keloid, pulmonary fibrosis, hepatic fibrosis, cardiac fibrosis, renal fibrosis, mediastinal fibrosis, retroperitoneal cavity fibrosis, bone marrow fibrosis, scleroderma or systemic sclerosis, or any combination thereof. In another aspect, the subject can be a human or non-human mammal.

In one aspect, disclosed herein is a rational approach for combining drugs of different classes to target different cellular pathways and/or different points within the same cellular pathway that independently contribute to keloid disease. Further in this aspect, the disclosed approach blocks multiple keloid promoting pathways simultaneously to provide synergistic therapeutic benefits.

In an aspect, the flexibility of the disclosed approach offers the possibility of using alternative combinations of drugs for patients who do not respond to the first drug combination. In another aspect, local administration combined with lower doses of individual drugs when used in combination results in minimal side effects. In still another aspect, simultaneous targeting of multiple pathways minimizes the potential for developing resistance to therapy.

In one aspect, the serine protease inhibitor targeting SERPINE1 (PAI1) can be selected from tiplaxtinin, annonacinone, aleplasinin, diaplasinin, CDE-096, AZ3976, TM5275, TM5007, TM5441, ACT001, a natural compound including, but not limited to, toddalolactone, loureirin B, embelin, or geodin, antibodies and nanobodies targeting SERPINE1, or any combination thereof. In another aspect, the HAT inhibitor can be natural or synthetic and can be selected from curcumin, garcinol, anacardic acid, C646, CPTH2, A485, or any combination thereof. In still another aspect, the HDAC inhibitor can be selected from FDA approved valproic acid, sodium valproate, Panobinostat, Belinostat, Vorinostat, Romidepsin, preclinical Tucidinostat or Entinostat, or any combination thereof. In yet another aspect, the IGF inhibitor can be chromeceptin Linsitinib, Ceritinib, a natural product such as, for example, ginsenoside or picropodophyllin, or any combination thereof. In one aspect, the anti-hypertensive agent can be selected from verapamil, minoxidil, hydralazine, nitroglycerin, amlodipine, diltiazem, losartan, telmisartan, captopril, lisinopril, atenolol, propranolol, prazosin, hydrochlorothiazide, furosemide, spironolactone, or any combination thereof. In one aspect, and without wishing to be bound by theory, minoxidil can act as a vasodilator, but is also a PLOD2 inhibitor (where PLOD2 is procollagen-lysine, 2-oxoglutarate 5-dioxygenase 2). Thus, in some aspects, minoxidil can exhibit synergistic effects when used in combination with another anti-hypertensive agent such as, for example, a calcium channel blocker including, but not limited to, verapamil.

In an aspect, the disclosed compositions and methods can include a topoisomerase II inhibitor such as, for example, mitoxantrone. In another aspect, mitoxantrone is, in some cases, useful as an antibiotic and as a treatment for multiple sclerosis.

In another aspect, the disclosed compositions and methods can include a tyrosine kinase inhibitor such as, for example, nintedanib. In a further aspect, nintedanib is typically used for the treatment of idiopathic pulmonary fibrosis and is FDA approved for treatment of chronic fibrosing interstitial lung diseases. In a further aspect, nintedanib targets various growth factors including, but not limited to, vascular endothelial growth factor receptor, fibroblast growth factor receptor, and platelet derived growth factor receptor.

In still another aspect, the disclosed compositions and methods can include a compound that inhibits TGF-β signaling such as, for example, pirfenidone, A 83-01, SB 525334, or any combination thereof. In another aspect, the TGF-β signaling inhibitors include SMAD signaling inhibitors. Further in this aspect, the composition can also downregulate procollagens such as, for example, procollagen I and procollagen II. In one aspect, pirfenidone is FDA approved for the treatment of idiopathic pulmonary fibrosis. In some countries pirfenidone has also been approved for treatment of scars and fibrotic tissue. In one aspect, TGF-β signaling is understood to be a major contributor to keloid formation, but has not previously been effectively targeted for keloid therapy due to other essential functions this pathway performs in cell physiology. In one aspect, TGF-β signaling inhibitors used in combination with other drugs belonging to different classes can reduce the dosage required for individual inhibitors and thus avoid adverse effects while still providing therapeutic benefits due to synergistic effects among different classes of drugs.

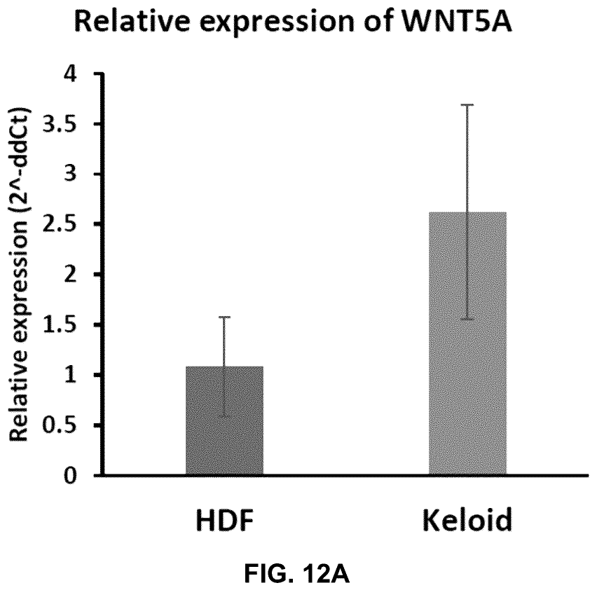

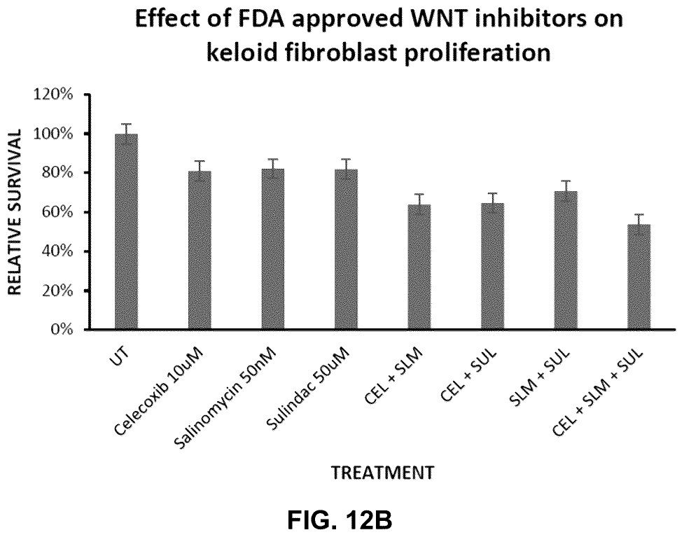

In one aspect, the disclosed compositions and methods include a WNT pathway inhibitor. In a further aspect, the WNT pathway has been found to be an important contributor to keloid disease in a transcriptomic analysis (FIG. 12A; see also Example 4). In another aspect, the WNT pathway inhibitor can be pre-clinical or FDA approved and can be selected from sulindac, celecoxib, salinomycin, CCT251545, Wnt-C59, zamaporvint, Wnt pathway inhibitor 3, 15-oxospiramilactone, CK2-In-9, Box5, Box5 TFA, TNKS-2-IN-1, hCA/Wnt/β-catenin-IN-1, MSAB, SSTC3, FzM1, ABC99, IWP-4, EMT inhibitor-1, IWR-1, FH535, iCRT3, pyrvinium pamoate, Pin1 modulator 1, IWP-3, WIC1, KYA1797K, G244-LM, IWP-12, carboxylesterase-IN-2, carboxylesterase-IN-3, Wnt pathway inhibitor 4, IM-12, coronaridine, TWS119, LGK974, WIKI4, PNU-74654, wogonin, TWS119 TFA, IWP-2, longdaysin, JW67, GNF-6231, teplinovivint, OM-153, YW1128, iCRT14, NSC668036, SEN461, TNKS-IN-2, YS2036, sempervivine nitrate, Notum-IN-1, atranorin, KY02111, ipvivint, CCT031374 hydrobromide, Adavivint, TNIK-IN-5, M435-1279, progdigiosin, exo-IWR-1, ME-143, echinacoside, prodigiosin hydrochloride, Porcn-IN-2, KY1022, hematein, gigantol isomer-1, WAY 316606, CDK8-IN-11, triptonide, NLS-StAx-h, GSK-3β inhibitor 8, ginkgetin, KY 02327, QS11, β-catenin-IN-7, NNGH, DK419, combretastatin A-1, TC-E 5001, UU-TO2, SGC-AAK1-1, YB-0158, iCRT-5, ID-8, 2-hydroxycinnamaldehyde, AV023, cardamonin, CWP232228, SRI 37892, FIDA S-3, windorphen, β-catenin-IN-37, cardinogen 1, E722-2648, vantinctumab, mirodenafil, KY-05009, specnuezhenide, KY 19382, laduviglusib, PDE5-IN-3, NSC260594, or any combination thereof.

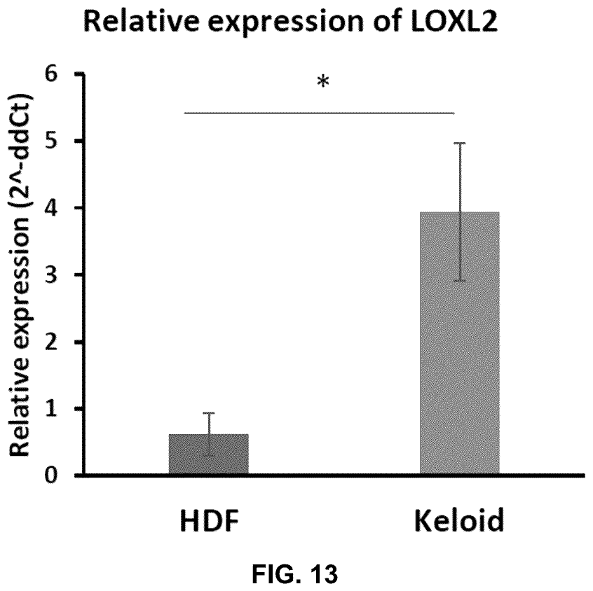

In still another aspect, the disclosed compositions include a lysyl oxidase-like 2 or lysyl oxidase-like 3 inhibitor. In one aspect, the lysyl oxidase-like 2 or lysyl oxidase-like 3 inhibitor can be PXS-5153A monohydrochloride. In an aspect, lysyl oxidase-like 2 or lysyl oxidase-like 3 are required for collagen maturation and contribute to keloid disease based on transcriptomic analysis (FIG. 13 and Example 4).

Various dosages are contemplated for the disclosed pharmaceutical compositions. In one aspect, in a topical pharmaceutical composition disclosed herein, the concentration of nintedanib can be from about 5 nM to about 75 nM, from about 10 nM to about 50 nM, or from about 35 nM to about 50 nM. In another aspect, the concentration of pirfenidone can be from about 0.1 mM to about 1.75 mM, or from about 0.25 mM to about 1.5 mM, or from about 0.5 mM to about 1 mM. In still another aspect, the concentration of minoxidil can be from about 5 μM to about 50 μM, from about 10 μM to about 40 μM, or from about 25 μM to about 30 μM. In one aspect, the concentration of mitoxantrone can be from about 1 nM to about 10 nM, from about 2 nM to about 8 nM, or from about 4 nM to about 5 nM. In still another aspect, the concentration of verapamil can be from about 50 UM to about 150 μM, from about 75 μM to about 125 μM, or from about 80 UM to about 100 μM. In still another aspect, the concentration of vorinostat in a disclosed composition can be from about 100 nM to about 350 nM, from about 150 nM to about 250 nM, or from about 150 nM to about 200 nM. In another aspect, the concentration of celecoxib in a disclosed composition can be from about 5 UM to about 25 UM, from about 10 UM to about 20 μM, or from about 10 UM to about 15 μM. In another aspect, the concentration of salinomycin in a disclosed composition can be from about 25 nM to about 100 nM, from about 25 nM to about 75 nM, or from about 50 nM to about 75 nM. In still another aspect, the concentration of sulindac in a disclosed composition can be from about 25 UM to about 100 UM, from about 25 UM to about 75 UM, or from about 50 μM to about 75 μM. In an aspect, when the compositions include triamcinolone acetonide, or when the triamcinolone acetonide is administered separately from the compositions, the concentration of triamcinolone acetonide can be from about 0.5 μM to about 10 μM, from about 1 μM to about 7.5 μM, or from about 1 μM to about 5 μM.

In one aspect, in the disclosed methods, superficial radiation therapy can also be administered to the subject harboring keloids, simultaneously or sequentially with the one or more therapeutic agents. In some aspects, higher doses of radiation that penetrate deeper into the tissues such as, for example, those used in cancer therapy, may be used for fibrotic disorders of the internal organs in conjunction with use of the therapeutic agents disclosed herein. Further in this aspect, superficial radiation therapy can be administered in a single dose at from about 8 Gy to about 10 Gy In a further aspect, the radiation dosage can be about 8, 8.5, 9, 9.5, or 10 Gy, or a combination of any of the foregoing values, or a range encompassing any of the foregoing values. In one aspect, radiation therapy can be administered before or after treatment with the disclosed compositions, or can be administered simultaneously with the disclosed compositions.

In an aspect, a single dose of superficial (i.e., skin deep) radiation at from about 8 to about 10 Gy is between 5- and 10-fold lower than the cumulative radiation doses delivered in multiple fractions typically used in cancer therapy. In a further aspect, radiation energies are typically higher in cancer therapy than in the disclosed methods, due to the need of radiation in cancer therapy to deeply penetrate to the site of the tumor. In a still further aspect, radiation therapy for cancer typically results in more collateral damage than the superficial radiation therapy used for keloid treatment. In one aspect, higher radiation dosages than those disclosed herein do not offer any additional benefit for keloid treatment. In still another aspect, radiation therapy has synergistic effects with the disclosed pharmaceutical compositions.

In still another aspect, the disclosed methods can further include performing surgical resection, before or after administering the therapeutic agents. In one aspect, the method further includes administering at least one additional therapeutic agent to the subject. In an aspect, the therapeutic agent can be a steroid such as, for example, triamcinolone acetonide, betamethasone acetate, dexamethasone, or any combination thereof. In some aspects, the steroids can exhibit synergistic effects in combination with the other therapeutic agents disclosed herein for treating steroid-responsive keloid patients as well as other fibrotic diseases and disorders.

Specific, non-limiting example combinations of therapeutic agents include chromeceptin and minoxidil, chromeceptin and tiplaxtinin, chromeceptin and vorinostat, minoxidil and verapamil, and minoxidil and tiplaxtinin.

Also disclosed herein are compositions including two or more of a SERPINE1 (PAI1) serine protease inhibitor, a histone acetyltransferase (HAT) inhibitor, a histone deacetylase (HDAC) inhibitor, an insulin-like growth factor (IGF) inhibitor, an anti-hypertensive agent, or any combination thereof, wherein the SERPINE1 serine protease inhibitor, HAT inhibitor, HDAC inhibitor, IGF inhibitor, anti-hypertensive agent, a topoisomerase II inhibitor, a tyrosine kinase inhibitor, an agent that inhibits TGF-β signaling, a WNT pathway inhibitor, and/or a lysyl oxidase-like 2 or lysyl oxidase-like 3 inhibitor can be selected from the lists presented herein, and provided that the compositions do not include both a HAT inhibitor and an HDAC inhibitor simultaneously. In some aspects, the compositions can include three or four of the disclosed therapeutic agents. In another aspect, the compositions can include at least two anti-hypertensive agents.

In one example composition, the IGF inhibitor is chromeceptin and the anti-hypertensive agent is minoxidil. In another example composition, the IGF inhibitor is chromeceptin and the SERPINE1 serine protease inhibitor is tiplaxtinin. In still another example composition, the IGF inhibitor is chromeceptin and the HDAC inhibitor can be vorinostat. In another example composition, the anti-hypertensive agent is minoxidil and the SERPINE1 serine protease inhibitor can be tiplaxtinin. In one aspect, when two anti-hypertensive agents are included in the compositions, the two anti-hypertensive agents can be minoxidil and verapamil.

In one aspect, the one or more therapeutic agents can be pirfenidone and nintedanib, or can be verapamil and nintedanib, or can be mitoxantrone and nintedanib, or can be celecoxib and salinomycin, or celecoxib and sulindac, or can be salinomycin and sulindac. Exemplary compositions including three or more therapeutic agents include, but are not limited to, verapamil, mitoxantrone, and nintedanib in one aspect; vorinostat, mitoxantrone, and nintedanib in another aspect; minoxidil, mitoxantrone, and nintedanib; or celecoxib, salinomycin, and sulindac in yet another aspect. However, other combinations of disclosed therapeutic agents are contemplated and should be considered disclosed.

In one aspect, the one or more therapeutic agents can be or include nintedanib and the compositions can also include triamcinolone acetonide. In an alternative aspect, triamcinolone acetonide can be administered in combination with pirfenidone and nintedanib, mitoxantrone and nintedanib, or verapamil and nintedanib. Other combinations of steroid and therapeutic agents discussed herein are contemplated and should also be considered disclosed. In a further aspect, the steroid can be included in the same pharmaceutical formulation as the one or more therapeutic agents, or can be included in a separate pharmaceutical composition. In still another aspect, the steroid can be administered simultaneously with the one or more therapeutic agents or sequentially before or after the one or more therapeutic agents.

In one aspect, the disclosed compositions further include a pharmaceutically acceptable carrier or diluent such as, for example, petrolatum, cetyl alcohol, glyceryl monostearate, lanolin, stearic acid, methylcellulose, carboxymethylcellulose, stearyl alcohol, a carbomer, a polyethylene oxide, a polyoxyethylene-polyoxypropylene copolymer, polyvinylalcohol, hydroxypropyl cellulose, hydroxyethyl cellulose, hydroxypropyl methylcellulose, hydroxypropyl methylcellulose phthalate, gum tragacanth, xanthan gum, sodium alginate, gelatin, ethyl alcohol, glycerin, water, or any combination thereof. In another aspect, for keloid therapy, the composition can be administered intralesionally via injection, or topically in a formulation such as, for example, an ointment, lotion, cream, paste, gel, spray, or foam. For treatment of other fibrotic disorders involving organs, systemic administration of the compositions may be performed. In some aspects, intralesional administration of the compositions can be performed.

Also disclosed herein is a method for treating or delaying the onset, progression, or relapse of a fibrotic disorder in a subject, the method comprising administering to the subject a disclosed composition.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. The skilled artisan will recognize many variants and adaptations of the aspects described herein. These variants and adaptations are intended to be included in the teachings of this disclosure and to be encompassed by the claims herein.

Although specific terms are employed herein, they are used in a generic and descriptive sense only and not for purposes of limitation.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure.

Any recited method can be carried out in the order of events recited or in any other order that is logically possible. That is, unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

While aspects of the present disclosure can be described and claimed in a particular statutory class, such as the system statutory class, this is for convenience only and one of skill in the art will understand that each aspect of the present disclosure can be described and claimed in any statutory class.

It is also to be understood that the terminology used herein is for the purpose of describing particular aspects only and is not intended to be limiting. Unless defined otherwise, all technical and scientific terms used herein have the same meaning as commonly understood by one of ordinary skill in the art to which the disclosed compositions and methods belong. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

Prior to describing the various aspects of the present disclosure, the following definitions are provided and should be used unless otherwise indicated. Additional terms may be defined elsewhere in the present disclosure.

### Definitions

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by,” “comprising,” “comprises,” “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Thus, for example, reference to “a drug,” “a fibroblast,” or “a biochemical pathway,” include, but are not limited to, mixtures, combinations, or sequences of two or more such drugs, fibroblasts, or biochemical pathways, and the like.

It should be noted that ratios, concentrations, amounts, and other numerical data can be expressed herein in a range format. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about,” it will be understood that the particular value forms a further aspect. For example, if the value “about 10” is disclosed, then “10” is also disclosed.

When a range is expressed, a further aspect includes from the one particular value and/or to the other particular value. For example, where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y.’ The range can also be expressed as an upper limit, e.g. ‘about x, y, z, or less’ and should be interpreted to include the specific ranges of ‘about x,’ ‘about y’, and ‘about z’ as well as the ranges of ‘less than x’, less than y’, and ‘less than z’. Likewise, the phrase ‘about x, y, z, or greater’ should be interpreted to include the specific ranges of ‘about x,’ ‘about y,’ and ‘about z’ as well as the ranges of ‘greater than x,’ greater than y,’ and ‘greater than z.’ In addition, the phrase “about ‘x’ to ‘y’”, where ‘x’ and ‘y’ are numerical values, includes “about ‘x’ to about ‘y’”.

It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., about 1%, about 2%, about 3%, and about 4%) and the sub-ranges (e.g., about 0.5% to about 1.1%; about 5% to about 2.4%; about 0.5% to about 3.2%, and about 0.5% to about 4.4%, and other possible sub-ranges) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a drug refers to an amount that is sufficient to achieve the desired improvement in the property modulated by the formulation component, e.g. achieving the desired reduction in keloid formation. The specific level in terms of wt % in a composition required as an effective amount will depend upon a variety of factors including the amount and type of drug, size, location, and age of the keloid, and the amount and type of any other drugs in combination with the drug in question.

As used herein, the terms “optional” or “optionally” means that the subsequently described event or circumstance can or cannot occur, and that the description includes instances where said event or circumstance occurs and instances where it does not.

As used herein, “administering” can refer to an administration that is topical, intralesional, or systemic by any acceptable means. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

As used herein, “parenteral administration” includes administration by bolus injection or infusion, as well as administration by intravenous, intramuscular, intraarterial, intrathecal, intracapsular, intraorbital, intracardiac, intradermal, intraperitoneal, transtracheal, subcutaneous, subcuticular, intraarticular, subcapsular subarachnoid, intraspinal, epidural, and intrasternal injection and infusion.

In a further aspect, the disclosed pharmaceutical compositions comprise a therapeutically effective amount of at least one disclosed therapeutic agent, at least one product of a disclosed method, or a pharmaceutically acceptable salt thereof as an active ingredient, a pharmaceutically acceptable carrier, optionally one or more other therapeutic agent, and optionally one or more adjuvant. The disclosed pharmaceutical compositions include those suitable for oral, rectal, topical, pulmonary, nasal, and parenteral administration, although the most suitable route in any given case will depend on the particular host, and nature and severity of the conditions for which the active ingredient is being administered. In a further aspect, the disclosed pharmaceutical composition can be formulated to allow administration orally, nasally, via inhalation, parenterally, paracancerally, transmucosally, transdermally, intramuscularly, intravenously, intradermally, subcutaneously, intraperitoneally, intraventricularly, intracranially, and intratumorally.

As used herein, “therapeutic agent” can refer to any substance, compound, molecule, and the like, which can be biologically active or otherwise can induce a pharmacologic, immunogenic, biologic and/or physiologic effect on a subject to which it is administered to by local and/or systemic action. A therapeutic agent can be a primary active agent, or in other words, the component(s) of a composition to which the whole or part of the effect of the composition is attributed. A therapeutic agent can be a secondary therapeutic agent, or in other words, the component(s) of a composition to which an additional part and/or other effect of the composition is attributed. The term therefore encompasses those compounds or chemicals traditionally regarded as drugs, vaccines, and biopharmaceuticals including molecules such as proteins, peptides, hormones, nucleic acids, gene constructs and the like.

The pharmaceutical compositions disclosed herein comprise a compound of the present disclosure (or pharmaceutically acceptable salts thereof) as an active ingredient, a pharmaceutically acceptable carrier, and optionally one or more additional therapeutic agents. In various aspects, the disclosed pharmaceutical compositions can include a pharmaceutically acceptable carrier and a disclosed compound, or a pharmaceutically acceptable salt thereof. In a further aspect, a disclosed compound, or pharmaceutically acceptable salt thereof, can also be included in a pharmaceutical composition in combination with one or more other therapeutically active compounds. The instant compositions include compositions suitable for topical administration. The pharmaceutical compositions can be conveniently presented in unit dosage form or another dosage form and prepared by any of the methods well known in the art of pharmacy.

Techniques and compositions for making dosage forms useful for materials and methods described herein are described, for example, in the following references: Modern Pharmaceutics, Chapters 9 and 10 (Banker & Rhodes, Editors, 1979); Pharmaceutical Dosage Forms: Tablets (Lieberman et al., 1981); Ansel, Introduction to Pharmaceutical Dosage Forms 2nd Edition (1976); Remington's Pharmaceutical Sciences, 17th ed. (Mack Publishing Company, Easton, Pa., 1985); Advances in Pharmaceutical Sciences (David Ganderton, Trevor Jones, Eds., 1992); Advances in Pharmaceutical Sciences Vol 7. (David Ganderton, Trevor Jones, James McGinity, Eds., 1995); Aqueous Polymeric Coatings for Pharmaceutical Dosage Forms (Drugs and the Pharmaceutical Sciences, Series 36 (James McGinity, Ed., 1989); Pharmaceutical Particulate Carriers: Therapeutic Applications: Drugs and the Pharmaceutical Sciences, Vol 61 (Alain Rolland, Ed., 1993); Drug Delivery to the Gastrointestinal Tract (Ellis Horwood Books in the Biological Sciences. Series in Pharmaceutical Technology; J. G. Hardy, S. S. Davis, Clive G. Wilson, Eds.); Modern Pharmaceutics Drugs and the Pharmaceutical Sciences, Vol 40 (Gilbert S. Banker, Christopher T. Rhodes, Eds.).

The compounds described herein are typically to be administered in admixture with suitable pharmaceutical diluents, excipients, extenders, or carriers (termed herein as a pharmaceutically acceptable carrier, or a carrier) suitably selected with respect to the intended form of administration and as consistent with conventional pharmaceutical practices. The deliverable compound will be in a form suitable for topical administration. Carriers include solids or liquids, and the type of carrier is chosen based on the type of administration being used. The compounds may be administered as a dosage that has a known quantity of the compound.

Because of the ease in administration, oral administration can be a preferred dosage form, and tablets and capsules represent the most advantageous oral dosage unit forms in which case solid pharmaceutical carriers are obviously employed. However, other dosage forms may be suitable depending upon clinical population (e.g., age and severity of clinical condition), solubility properties of the specific disclosed therapeutic agent used, and the like. Accordingly, the disclosed therapeutic agents can be used in oral dosage forms such as pills, powders, granules, elixirs, tinctures, suspensions, syrups, and emulsions. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs, and solutions; while carriers such as starches, sugars, microcrystalline cellulose, diluents, granulating agents, lubricants, binders, disintegrating agents, and the like can be used to form oral solid preparations such as powders, capsules, and tablets. Because of their ease of administration, tablets and capsules are the preferred oral dosage units whereby solid pharmaceutical carriers are employed. Optionally, tablets can be coated by standard aqueous or nonaqueous techniques.

The disclosed pharmaceutical compositions in an oral dosage form can comprise one or more pharmaceutical excipient and/or additive. Non-limiting examples of suitable excipients and additives include gelatin, natural sugars such as raw sugar or lactose, lecithin, pectin, starches (for example corn starch or amylose), dextran, polyvinyl pyrrolidone, polyvinyl acetate, gum arabic, alginic acid, tylose, talcum, lycopodium, silica gel (for example colloidal), cellulose, cellulose derivatives (for example cellulose ethers in which the cellulose hydroxy groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate), fatty acids as well as magnesium, calcium or aluminum salts of fatty acids with 12 to 22 carbon atoms, in particular saturated (for example stearates), emulsifiers, oils and fats, in particular vegetable (for example, peanut oil, castor oil, olive oil, sesame oil, cottonseed oil, corn oil, wheat germ oil, sunflower seed oil, cod liver oil, in each case also optionally hydrated); glycerol esters and polyglycerol esters of saturated fatty acids C12H2402 to C18H3602 and their mixtures, it being possible for the glycerol hydroxy groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentaerythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other auxiliary substances useful in preparing an oral dosage form are those which cause disintegration (so-called disintegrants), such as: cross-linked polyvinyl pyrrolidone, sodium carboxymethyl starch, sodium carboxymethyl cellulose or microcrystalline cellulose. Conventional coating substances may also be used to produce the oral dosage form. Those that may for example be considered are: polymerizates as well as copolymerizates of acrylic acid and/or methacrylic acid and/or their esters; copolymerizates of acrylic and methacrylic acid esters with a lower ammonium group content (for example EudragitR RS), copolymerizates of acrylic and methacrylic acid esters and trimethyl ammonium methacrylate (for example EudragitR RL); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine.

Plasticizing agents that may be considered as coating substances in the disclosed oral dosage forms are: citric and tartaric acid esters (acetyl-triethyl citrate, acetyl tributyl-, tributyl-, triethyl-citrate); glycerol and glycerol esters (glycerol diacetate, -triacetate, acetylated monoglycerides, castor oil); phthalic acid esters (dibutyl-, diamyl-, diethyl-, dimethyl-, dipropyl-phthalate), di-(2-methoxy- or 2-ethoxyethyl)-phthalate, ethylphthalyl glycolate, butylphthalylethyl glycolate and butylglycolate; alcohols (propylene glycol, polyethylene glycol of various chain lengths), adipates (diethyladipate, di-(2-methoxy- or 2-ethoxyethyl)-adipate; benzophenone; diethyl- and diburylsebacate, dibutylsuccinate, dibutyltartrate; diethylene glycol dipropionate; ethyleneglycol diacetate, -dibutyrate, -dipropionate; tributyl phosphate, tributyrin; polyethylene glycol sorbitan monooleate (polysorbates such as Polysorbar 50); sorbitan monooleate.

Moreover, suitable binders, lubricants, disintegrating agents, coloring agents, flavoring agents, flow-inducing agents, and melting agents may be included as carriers. The pharmaceutical carrier employed can be, for example, a solid, liquid, or gas. Examples of solid carriers include, but are not limited to, lactose, terra alba, sucrose, glucose, methylcellulose, dicalcium phosphate, calcium sulfate, mannitol, sorbitol talc, starch, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In various aspects, a binder can include, for example, starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. In a further aspect, a disintegrator can include, for example, starch, methyl cellulose, agar, bentonite, xanthan gum, and the like.

In various aspects, an oral dosage form, such as a solid dosage form, can comprise a disclosed therapeutic agent that is attached to polymers as targetable drug carriers or as a prodrug. Suitable biodegradable polymers useful in achieving controlled release of a drug include, for example, polylactic acid, polyglycolic acid, copolymers of polylactic and polyglycolic acid, caprolactones, polyhydroxy butyric acid, polyorthoesters, polyacetals, polydihydropyrans, polycyanoacylates, and hydrogels, preferably covalently crosslinked hydrogels.

Tablets may contain the active ingredient in admixture with non-toxic pharmaceutically acceptable excipients which are suitable for the manufacture of tablets. These excipients may be, for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid, or talc. The tablets may be uncoated or they may be coated by known techniques to delay disintegration and absorption in the gastrointestinal tract and thereby provide a sustained action over a longer period.

A tablet containing a disclosed therapeutic agent can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

In various aspects, a solid oral dosage form, such as a tablet, can be coated with an enteric coating to prevent ready decomposition in the stomach. In various aspects, enteric coating agents include, but are not limited to, hydroxypropylmethylcellulose phthalate, methacrylic acid-methacrylic acid ester copolymer, polyvinyl acetate-phthalate, and cellulose acetate phthalate. Akihiko Hasegawa “Application of solid dispersions of Nifedipine with enteric coating agent to prepare a sustained-release dosage form” Chem. Pharm. Bull. 33:1615-1619 (1985). Various enteric coating materials may be selected on the basis of testing to achieve an enteric coated dosage form designed ab initio to have a preferable combination of dissolution time, coating thicknesses and diametral crushing strength (e.g., see S. C. Porter et al. “The Properties of Enteric Tablet Coatings Made From Polyvinyl Acetate-phthalate and Cellulose acetate Phthalate”, J. Pharm. Pharmacol. 22:42p (1970)). In a further aspect, the enteric coating may comprise hydroxypropyl-methylcellulose phthalate, methacrylic acid-methacrylic acid ester copolymer, polyvinyl acetate-phthalate, and cellulose acetate phthalate.

In various aspects, an oral dosage form can be a solid dispersion with a water soluble or a water insoluble carrier. Examples of water soluble or water insoluble carrier include, but are not limited to, polyethylene glycol, polyvinylpyrrolidone, hydroxypropylmethyl-cellulose, phosphatidylcholine, polyoxyethylene hydrogenated castor oil, hydroxypropylmethylcellulose phthalate, carboxymethylethylcellulose, or hydroxypropylmethylcellulose, ethyl cellulose, or stearic acid.

In various aspects, an oral dosage form can be in a liquid dosage form, including those that are ingested, or alternatively, administered as a mouth wash or gargle. For example, a liquid dosage form can include aqueous suspensions, which contain the active materials in admixture with excipients suitable for the manufacture of aqueous suspensions. In addition, oily suspensions may be formulated by suspending the active ingredient in a vegetable oil, for example arachis oil, olive oil, sesame oil or coconut oil, or in a mineral oil such as liquid paraffin. Oily suspensions may also contain various excipients. The pharmaceutical compositions of the present disclosure may also be in the form of oil-in-water emulsions, which may also contain excipients such as sweetening and flavoring agents.

For the preparation of solutions or suspensions it is, for example, possible to use water, particularly sterile water, or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulfoxide, triglycerides and the like.

In the case of a liquid dosage form such as a drinkable solutions, the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), diethylene glycol monoethyl ether, 1,2-propylene glycol, organic amides, for example amides of aliphatic C1-C6-carboxylic acids with ammonia or primary, secondary or tertiary C1-C4-amines or C1-C4-hydroxy amines such as urea, urethane, acetamide, N-methyl acetamide, N,N-diethyl acetamide, N,N-dimethyl acetamide, lower aliphatic amines and diamines with 2-6 carbon atoms, such as ethylene diamine, hydroxyethyl theophylline, tromethamine (for example as 0.1 to 20% aqueous solution), aliphatic amino acids.

In preparing the disclosed liquid dosage form can comprise solubilizers and emulsifiers such as the following non-limiting examples can be used: polyvinyl pyrrolidone, sorbitan fatty acid esters such as sorbitan trioleate, phosphatides such as lecithin, acacia, tragacanth, polyoxyethylated sorbitan monooleate and other ethoxylated fatty acid esters of sorbitan, polyoxyethylated fats, polyoxyethylated oleotriglycerides, linolizated oleotriglycerides, polyethylene oxide condensation products of fatty alcohols, alkylphenols or fatty acids or also 1-methyl-3-(2-hydroxyethyl)imidazolidone-(2). In this context, polyoxyethylated means that the substances in question contain polyoxyethylene chains, the degree of polymerization of which generally lies between 2 and 40 and in particular between 10 and 20. Polyoxyethylated substances of this kind may for example be obtained by reaction of hydroxyl group-containing compounds (for example mono- or diglycerides or unsaturated compounds such as those containing oleic acid radicals) with ethylene oxide (for example 40 μMol ethylene oxide per 1 μMol glyceride). Examples of oleotriglycerides are olive oil, peanut oil, castor oil, sesame oil, cottonseed oil, corn oil. See also Dr. H. P. Fiedler “Lexikon der Hillsstoffe für Pharmazie, Kostnetik und angrenzende Gebiete” 1971, pages 191-195.

In various aspects, a liquid dosage form can further comprise preservatives, stabilizers, buffer substances, flavor correcting agents, sweeteners, colorants, antioxidants, and complex formers and the like. Complex formers which may be for example be considered are: chelate formers such as ethylene diamine retrascetic acid, nitrilotriacetic acid, diethylene triamine pentacetic acid and their salts.

It may optionally be necessary to stabilize a liquid dosage form with physiologically acceptable bases or buffers to a pH range of approximately 6 to 9. Preference may be given to as neutral or weakly basic a pH value as possible (up to pH 8).

In order to enhance the solubility and/or the stability of a disclosed therapeutic agent in a disclosed liquid dosage form, a parenteral injection form, or an intravenous injectable form, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also co-solvents such as alcohols may improve the solubility and/or the stability of the compounds according to the present disclosure in pharmaceutical compositions.

In various aspects, a disclosed liquid dosage form, a parenteral injection form, or an intravenous injectable form can further comprise liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

Pharmaceutical compositions of the present disclosure suitable injection, such as parenteral administration, such as intravenous, intramuscular, or subcutaneous administration. Pharmaceutical compositions for injection can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present disclosure suitable for parenteral administration can include sterile aqueous or oleaginous solutions, suspensions, or dispersions. Furthermore, the compositions can be in the form of sterile powders for the extemporaneous preparation of such sterile injectable solutions or dispersions. In some aspects, the final injectable form is sterile and must be effectively fluid for use in a syringe. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. In some aspects, a disclosed parenteral formulation can comprise about 0.01-0.1 M, e.g. about 0.05 μM, phosphate buffer. In a further aspect, a disclosed parenteral formulation can comprise about 0.9% saline.

In various aspects, a disclosed parenteral pharmaceutical composition can comprise pharmaceutically acceptable carriers such as aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include but not limited to water, alcoholic/aqueous solutions, emulsions, or suspensions, including saline and buffered media. Parenteral vehicles can include mannitol, normal serum albumin, sodium chloride solution, Ringer's dextrose, dextrose and sodium chloride, lactated Ringer's, and fixed oils. Intravenous vehicles include fluid and nutrient replenishers, electrolyte replenishers such as those based on Ringer's dextrose, and the like. Preservatives and other additives may also be present, such as, for example, antimicrobials, antioxidants, chelating agents, inert gases, and the like. In a further aspect, a disclosed parenteral pharmaceutical composition can comprise may contain minor amounts of additives such as substances that enhance isotonicity and chemical stability, e.g., buffers and preservatives. Also contemplated for injectable pharmaceutical compositions are solid form preparations that are intended to be converted, shortly before use, to liquid form preparations. Furthermore, other adjuvants can be included to render the formulation isotonic with the blood of the subject or patient.

In addition to the pharmaceutical compositions described herein above, the disclosed therapeutic agents can also be formulated as a depot preparation. Such long acting formulations can be administered by implantation (e.g., subcutaneously or intramuscularly) or by intramuscular injection. Thus, for example, the compounds can be formulated with suitable polymeric or hydrophobic materials (e.g., as an emulsion in an acceptable oil) or ion exchange resins, or as sparingly soluble derivatives, e.g., as a sparingly soluble salt.

Pharmaceutical compositions of the present disclosure can be in a form suitable for topical administration. As used herein, the phrase “topical application” means administration onto a biological surface, whereby the biological surface includes, for example, a skin area (e.g., hands, forearms, elbows, legs, face, nails, anus, and genital areas) or a mucosal membrane. By selecting the appropriate carrier and optionally other ingredients that can be included in the composition, as is detailed herein below, the compositions of the present invention may be formulated into any form typically employed for topical application. A topical pharmaceutical composition can be in a form of a cream, an ointment, a paste, a gel, a lotion, milk, a suspension, an aerosol, a spray, foam, a dusting powder, a pad, and a patch. Further, the compositions can be in a form suitable for use in transdermal devices. These formulations can be prepared, utilizing a compound of the present disclosure, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot-on, as an ointment.

Ointments are semisolid preparations, typically based on petrolatum or petroleum derivatives. The specific ointment base to be used is one that provides for optimum delivery for the active agent chosen for a given formulation, and, preferably, provides for other desired characteristics as well (e.g., emollience). As with other carriers or vehicles, an ointment base should be inert, stable, nonirritating and nonsensitizing. As explained in Remington: The Science and Practice of Pharmacy, 19th Ed., Easton, Pa.: Mack Publishing Co. (1995), pp. 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, cetyl alcohol, glyceryl monostearate, lanolin and stearic acid. Preferred water-soluble ointment bases are prepared from polyethylene glycols of varying molecular weight.

Lotions are preparations that are to be applied to the skin surface without friction. Lotions are typically liquid or semiliquid preparations in which solid particles, including the active agent, are present in a water or alcohol base. Lotions are typically preferred for treating large body areas, due to the ease of applying a more fluid composition. Lotions are typically suspensions of solids, and oftentimes comprise a liquid oily emulsion of the oil-in-water type. It is generally necessary that the insoluble matter in a lotion be finely divided. Lotions typically contain suspending agents to produce better dispersions as well as compounds useful for localizing and holding the active agent in contact with the skin, such as methylcellulose, sodium carboxymethyl-cellulose, and the like.

Creams are viscous liquids or semisolid emulsions, either oil-in-water or water-in-oil. Cream bases are typically water-washable, and contain an oil phase, an emulsifier, and an aqueous phase. The oil phase, also called the “internal” phase, is generally comprised of petrolatum and/or a fatty alcohol such as cetyl or stearyl alcohol. The aqueous phase typically, although not necessarily, exceeds the oil phase in volume, and generally contains a humectant. The emulsifier in a cream formulation is generally a nonionic, anionic, cationic, or amphoteric surfactant. Reference may be made to Remington: The Science and Practice of Pharmacy, supra, for further information.

Pastes are semisolid dosage forms in which the bioactive agent is suspended in a suitable base. Depending on the nature of the base, pastes are divided between fatty pastes or those made from a single-phase aqueous gel. The base in a fatty paste is generally petrolatum, hydrophilic petrolatum and the like. The pastes made from single-phase aqueous gels generally incorporate carboxymethylcellulose or the like as a base. Additional reference may be made to Remington: The Science and Practice of Pharmacy, for further information.

Gel formulations are semisolid, suspension-type systems. Single-phase gels contain organic macromolecules distributed substantially uniformly throughout the carrier liquid, which is typically aqueous, but also, preferably, contain an alcohol and, optionally, an oil. Preferred organic macromolecules, i.e., gelling agents, are crosslinked acrylic acid polymers such as the family of carbomer polymers, e.g., carboxypolyalkylenes that may be obtained commercially under the trademark Carbopol™. Other types of preferred polymers in this context are hydrophilic polymers such as polyethylene oxides, polyoxyethylene-polyoxypropylene copolymers and polyvinylalcohol; modified cellulose, such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Sprays generally provide the active agent in an aqueous and/or alcoholic solution which can be misted onto the skin for delivery. Such sprays include those formulated to provide for concentration of the active agent solution at the site of administration following delivery, e.g., the spray solution can be primarily composed of alcohol or other like volatile liquid in which the active agent can be dissolved. Upon delivery to the skin, the carrier evaporates, leaving concentrated active agent at the site of administration.

Foam compositions are typically formulated in a single or multiple phase liquid form and housed in a suitable container, optionally together with a propellant which facilitates the expulsion of the composition from the container, thus transforming it into a foam upon application. Other foam forming techniques include, for example the “Bag-in-a-can” formulation technique. Compositions thus formulated typically contain a low-boiling hydrocarbon, e.g., isopropane. Application and agitation of such a composition at the body temperature cause the isopropane to vaporize and generate the foam, in a manner similar to a pressurized aerosol foaming system. Foams can be water-based or aqueous alkanolic, but are typically formulated with high alcohol content which, upon application to the skin of a user, quickly evaporates, driving the active ingredient through the upper skin layers to the site of treatment.

Skin patches typically comprise a backing, to which a reservoir containing the active agent is attached. The reservoir can be, for example, a pad in which the active agent or composition is dispersed or soaked, or a liquid reservoir. Patches typically further include a frontal water permeable adhesive, which adheres and secures the device to the treated region. Silicone rubbers with self-adhesiveness can alternatively be used. In both cases, a protective permeable layer can be used to protect the adhesive side of the patch prior to its use. Skin patches may further comprise a removable cover, which serves for protecting it upon storage.

Examples of patch configuration which can be utilized with the present invention include a single-layer or multi-layer drug-in-adhesive systems which are characterized by the inclusion of the drug directly within the skin-contacting adhesive. In such a transdermal patch design, the adhesive not only serves to affix the patch to the skin, but also serves as the formulation foundation, containing the drug and all the excipients under a single backing film. In the multi-layer drug-in-adhesive patch a membrane is disposed between two distinct drug-in-adhesive layers or multiple drug-in-adhesive layers are incorporated under a single backing film. In some aspects, biopolymer adhesives that fix the patch to the patient's skin or other fibrotic organ can act as drug delivery mechanisms.

Examples of pharmaceutically acceptable carriers that are suitable for pharmaceutical compositions for topical applications include carrier materials that are well-known for use in the cosmetic and medical arts as bases for e.g., emulsions, creams, aqueous solutions, oils, ointments, pastes, gels, lotions, milks, foams, suspensions, aerosols and the like, depending on the final form of the composition. Representative examples of suitable carriers according to the present invention therefore include, without limitation, water, liquid alcohols, liquid glycols, liquid polyalkylene glycols, liquid esters, liquid amides, liquid protein hydrolysates, liquid alkylated protein hydrolysates, liquid lanolin, and lanolin derivatives, and like materials commonly employed in cosmetic and medicinal compositions. Other suitable carriers according to the present invention include, without limitation, alcohols, such as, for example, monohydric and polyhydric alcohols, e.g., ethanol, isopropanol, glycerol, sorbitol, 2-methoxyethanol, diethyleneglycol, ethylene glycol, hexyleneglycol, mannitol, and propylene glycol; ethers such as diethyl or dipropyl ether; polyethylene glycols and methoxypolyoxyethylenes (carbowaxes having molecular weight ranging from 200 to 20,000); polyoxyethylene glycerols, polyoxyethylene sorbitols, stearoyl diacetin, and the like.

Topical compositions of the present disclosure can, if desired, be presented in a pack or dispenser device, such as an FDA-approved kit, which may contain one or more unit dosage forms containing the active ingredient. The dispenser device may, for example, comprise a tube. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration for prescription drugs or of an approved product insert. Compositions comprising the topical composition of the invention formulated in a pharmaceutically acceptable carrier may also be prepared, placed in an appropriate container, and labeled for treatment of an indicated condition.

Pharmaceutical compositions of the present disclosure can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In one aspect, topical administration may be useful for avoiding systemic side effects. However, in another aspect, topical administration may only be achievable for skin fibrosis (e.g. keloids), while systemic or intra-organ administration would be required for other fibrotic disorders. In a further aspect, however, lower doses of therapeutic agents used in combination may be useful in reducing systemic side effects when systemic or intra-organ administration are required.

Unless otherwise specified, temperatures referred to herein are based on atmospheric pressure (i.e. one atmosphere).

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

### Aspects

The present disclosure can be described in accordance with the following numbered aspects, which should not be confused with the claims.

Aspect 1. A method for treating or delaying the onset, progression, or relapse of a fibrotic disorder in a subject, the method comprising administering to the subject one or more therapeutic agents comprising a SERPINE1 serine protease inhibitor, a histone acetyltransferase (HAT) inhibitor, a histone deacetylase (HDAC) inhibitor, an insulin-like growth factor (IGF) inhibitor, an anti-hypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that a HAT inhibitor and a HDAC inhibitor are not simultaneously administered to the subject.

Aspect 2. The method of aspect 1, wherein the fibrotic disorder comprises keloid, pulmonary fibrosis, hepatic fibrosis, cardiac fibrosis, renal fibrosis, mediastinal fibrosis, retroperitoneal cavity fibrosis, bone marrow fibrosis, scleroderma, or any combination thereof.

Aspect 3. The method of aspect 1 or 2, wherein the one or more therapeutic agents are administered topically, parenterally, intralesionally, orally, or any combination thereof.

Aspect 4. The method of any one of aspects 1-3, wherein the subject is a human or non-human mammal.

Aspect 5. The method of any one of aspects 1-4, wherein the SERPINE1 serine protease inhibitor comprises tiplaxtinin, annonacinone, aleplasinin, diaplasinin, CDE-096, AZ3976, TM5275, TM5007, TM5441, ACT001, toddalolactone, loureirin B, embelin, or geodin, antibodies or nanobodies targeting SERPINE1, or any combination thereof.

Aspect 6. The method of any one of aspects 1-5, wherein the HAT inhibitor is natural or synthetic.

Aspect 7. The method of any one of aspects 1-6, wherein the HAT inhibitor comprises curcumin, garcinol, anacardic acid, C646, CPTH2, A485, or any combination thereof.

Aspect 8. The method of any one of aspects 1-7, wherein the HDAC inhibitor comprises valproic acid, sodium valproate, Panobinostat, Belinostat, Vorinostat, Romidepsin, or any combination thereof.

Aspect 9. The method of any one of aspects 1-8, wherein the IGF inhibitor comprises chromeceptin Linsitinib, Ceritinib, ginsenoside, picropodophyllin, or any combination thereof.

Aspect 10. The method of any one of aspects 1-9, wherein the anti-hypertensive agent comprises verapamil, minoxidil, hydralazine, nitroglycerin, amlodipine, diltiazem, losartan, telmisartan, captopril, lisinopril, atenolol, propranolol, prazosin, hydrochlorothiazide, furosemide, spironolactone, or any combination thereof.

Aspect 11. The method of any one of aspects 1-10, wherein the topoisomerase II inhibitor comprises mitoxantrone.

Aspect 12. The method of any one of aspects 1-11, wherein the tyrosine kinase inhibitor comprises nintedanib.

Aspect 13. The method of any one of aspects 1-12, wherein the agent that inhibits TGF-β signaling comprises pirfenidone, A 83-01, SB 525334, or any combination thereof.

Aspect 14. The method of any one of aspects 1-13, wherein the WNT pathway inhibitor comprises sulindac, celecoxib, salinomycin, CCT251545, Wnt-C59, zamaporvint, Wnt pathway inhibitor 3, 15-oxospiramilactone, CK2-In-9, Box5, Box5 TFA, TNKS-2-IN-1, hCA/Wnt/β-catenin-IN-1, MSAB, SSTC3, FzM1, ABC99, IWP-4, EMT inhibitor-1, IWR-1, FH535, iCRT3, pyrvinium pamoate, Pin1 modulator 1, IWP-3, WIC1, KYA1797K, G244-LM, IWP-12, carboxylesterase-IN-2, carboxylesterase-IN-3, Wnt pathway inhibitor 4, IM-12, coronaridine, TWS119, LGK974, WIKI4, PNU-74654, wogonin, TWS119 TFA, IWP-2, longdaysin, JW67, GNF-6231, teplinovivint, OM-153, YW1128, iCRT14, NSC668036, SEN461, TNKS-IN-2, YS2036, sempervivine nitrate, Notum-IN-1, atranorin, KY02111, ipvivint, CCT031374 hydrobromide, Adavivint, TNIK-IN-5, M435-1279, progdigiosin, exo-IWR-1, ME-143, echinacoside, prodigiosin hydrochloride, Porcn-IN-2, KY1022, hematein, gigantol isomer-1, WAY 316606, CDK8-IN-11, triptonide, NLS-StAx-h, GSK-3B inhibitor 8, ginkgetin, KY 02327, QS11, β-catenin-IN-7, NNGH, DK419, combretastatin A-1, TC-E 5001, UU-TO2, SGC-AAK1-1, YB-0158, iCRT-5, ID-8, 2-hydroxycinnamaldehyde, AV023, cardamonin, CWP232228, SRI 37892, FIDA S-3, windorphen, β-catenin-IN-37, cardinogen 1, E722-2648, vantinctumab, mirodenafil, KY-05009, specnuezhenide, KY 19382, laduviglusib, PDE5-IN-3, NSC260594, or any combination thereof.

Aspect 15. The method of any one of aspects 1-14, wherein the lysyl oxidase-like 2 or lysyl oxidase-like 3 inhibitor comprises PXS-5153A monohydrochloride.

Aspect 16. The method of any one of aspects 1-15, wherein the fibrotic disorder comprises keloid and wherein the method further comprises administering superficial radiation therapy to the subject.

Aspect 17. The method of aspect 16, wherein the superficial radiation therapy is administered simultaneously or sequentially with the one or more therapeutic agents.

Aspect 18. The method of aspect 16 or 17, wherein the superficial radiation therapy is administered in a single dose.

Aspect 19. The method of aspect 18, wherein the single dose of radiation therapy is from about 8 Gy to about 10 Gy.

Aspect 20. The method of any one of aspects 1-19, further comprising performing surgical resection.

Aspect 21. The method of aspect 20, wherein surgical resection is performed before or after administering the one or more therapeutic agents.

Aspect 22. The method of any one of aspects 1-21, further comprising administering at least one additional therapeutic agent to the subject.

Aspect 23. The method of aspect 22, wherein the at least one additional therapeutic agent comprises a steroid.

Aspect 24. The method of aspect 23, wherein the steroid comprises triamcinolone acetonide, betamethasone acetate, dexamethasone, or any combination thereof.

Aspect 25. The method of any one of aspects 1-24, wherein the one or more therapeutic agents comprise chromeceptin and minoxidil.

Aspect 26. The method of any one of aspects 1-24, wherein the one or more therapeutic agents comprise chromeceptin and tiplaxtinin.

Aspect 27. The method of any one of aspects 1-24, wherein the one or more therapeutic agents comprise chromeceptin and vorinostat.

Aspect 28. The method of any one of aspects 1-24, wherein the one or more therapeutic agents comprise minoxidil and tiplaxtinin.

Aspect 29. The method of any one of aspects 1-24, wherein the one or more therapeutic agents comprise minoxidil and verapamil.

Aspect 30. The method of any one of aspects 1-24, wherein the one or more therapeutic agents comprise pirfenidone and nintedanib.

Aspect 31. The method of any one of aspects 1-24, wherein the one or more therapeutic agents comprise verapamil and nintedanib.

Aspect 32. The method of any one of aspects 1-24, wherein the one or more therapeutic agents comprise mitoxantrone and nintedanib.

Aspect 33. The method of any one of aspects 1-24, wherein the one or more therapeutic agents comprise verapamil, mitoxantrone, and nintedanib.

Aspect 34. The method of any one of aspects 1-24, wherein the one or more therapeutic agents comprise vorinostat, mitoxantrone, and nintedanib.

Aspect 35. The method of any one of aspects 1-24, wherein the one or more therapeutic agents comprise minoxidil, mitoxantrone, and nintedanib.

Aspect 36. The method of any one of aspects 1-24, wherein the one or more therapeutic agents comprise celecoxib and salinomycin.

Aspect 37. The method of any one of aspects 1-24, wherein the one or more therapeutic agents comprise celecoxib and sulindac.

Aspect 38. The method of any one of aspects 1-24, wherein the one or more therapeutic agents comprise salinomycin and sulindac.

Aspect 39. The method of any one of aspects 1-24, wherein the one or more therapeutic agents comprise celecoxib, salinomycin, and sulindac.

Aspect 40. The method of aspect 24, wherein the one or more therapeutic agents comprise nintedanib and the steroid comprises triamcinolone acetonide.

Aspect 41. The method of aspect 24, wherein the one or more therapeutic agents comprise pirfenidone and nintedanib and the steroid comprises triamcinolone acetonide.

Aspect 42. The method of aspect 24, wherein the one or more therapeutic agents comprise mitoxantrone and nintedanib and the steroid comprises triamcinolone acetonide.

Aspect 43. The method of aspect 24, wherein the one or more therapeutic agents comprise verapamil and nintedanib and the steroid comprises triamcinolone acetonide.

Aspect 44. The method of any one of aspects 1-43, wherein the method comprises administering at least three of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that the composition does not comprise both a HDAC inhibitor and a HAT inhibitor.

Aspect 45. The method of any one of aspects 1-44, wherein the method comprises administering at least four of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that the composition does not comprise both a HDAC inhibitor and a HAT inhibitor.

Aspect 46. A composition comprising two or more of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that the composition does not comprise both a HDAC inhibitor and a HAT inhibitor.

Aspect 47. The composition of aspect 46, wherein the SERPINE1 serine protease inhibitor comprises tiplaxtinin, annonacinone, aleplasinin, diaplasinin, CDE-096, AZ3976, TM5275, TM5007, TM5441, ACT001, toddalolactone, loureirin B, embelin, or geodin, antibodies or nanobodies targeting SERPINE1, or any combination thereof.

Aspect 48. The composition of aspect 46 or 47, wherein the HAT inhibitor is natural or synthetic.

Aspect 49. The composition of any one of aspects 46-48, wherein the HAT inhibitor comprises curcumin, garcinol, anacardic acid, C646, CPTH2, A485, or any combination thereof.

Aspect 50. The composition of any one of aspects 46-49, wherein the HDAC inhibitor comprises valproic acid, sodium valproate, Panobinostat, Belinostat, Vorinostat, Romidepsin, or any combination thereof.

Aspect 51. The composition of any one of aspects 46-50, wherein the IGF inhibitor comprises chromeceptin Linsitinib, Ceritinib, ginsenoside, picropodophyllin, or any combination thereof.

Aspect 52. The composition of any one of aspects 46-51, wherein the anti-hypertensive agent comprises verapamil, minoxidil, hydralazine, nitroglycerin, amlodipine, diltiazem, losartan, telmisartan, captopril, lisinopril, atenolol, propranolol, prazosin, hydrochlorothiazide, furosemide, spironolactone, or any combination thereof.

Aspect 53. The composition of any one of aspects 46-52, wherein the topoisomerase II inhibitor comprises mitoxantrone.

Aspect 54. The composition of any one of aspects 46-53, wherein the tyrosine kinase inhibitor comprises nintedanib.

Aspect 55. The composition of any one of aspects 46-54, wherein the agent that inhibits TGF-β signaling comprises pirfenidone, A 83-01, SB 525334, or any combination thereof.

Aspect 56. The composition of any one of aspects 46-55, wherein the WNT pathway inhibitor comprises sulindac, celecoxib, salinomycin, CCT251545, Wnt-C59, zamaporvint, Wnt pathway inhibitor 3, 15-oxospiramilactone, CK2-In-9, Box5, Box5 TFA, TNKS-2-IN-1, hCA/Wnt/β-catenin-IN-1, MSAB, SSTC3, FzM1, ABC99, IWP-4, EMT inhibitor-1, IWR-1, FH535, iCRT3, pyrvinium pamoate, Pin1 modulator 1, IWP-3, WIC1, KYA1797K, G244-LM, IWP-12, carboxylesterase-IN-2, carboxylesterase-IN-3, Wnt pathway inhibitor 4, IM-12, coronaridine, TWS119, LGK974, WIKI4, PNU-74654, wogonin, TWS119 TFA, IWP-2, longdaysin, JW67, GNF-6231, teplinovivint, OM-153, YW1128, iCRT14, NSC668036, SEN461, TNKS-IN-2, YS2036, sempervivine nitrate, Notum-IN-1, atranorin, KY02111, ipvivint, CCT031374 hydrobromide, Adavivint, TNIK-IN-5, M435-1279, progdigiosin, exo-IWR-1, ME-143, echinacoside, prodigiosin hydrochloride, Porcn-IN-2, KY1022, hematein, gigantol isomer-1, WAY 316606, CDK8-IN-11, triptonide, NLS-StAx-h, GSK-3B inhibitor 8, ginkgetin, KY 02327, QS11, β-catenin-IN-7, NNGH, DK419, combretastatin A-1, TC-E 5001, UU-TO2, SGC-AAK1-1, YB-0158, iCRT-5, ID-8, 2-hydroxycinnamaldehyde, AV023, cardamonin, CWP232228, SRI 37892, FIDA S-3, windorphen, β-catenin-IN-37, cardinogen 1, E722-2648, vantinctumab, mirodenafil, KY-05009, specnuezhenide, KY 19382, laduviglusib, PDE5-IN-3, NSC260594, or any combination thereof.

Aspect 57. The composition of any one of aspects 46-56, wherein the lysyl oxidase-like 2 or lysyl oxidase-like 3 inhibitor comprises PXS-5153A monohydrochloride.

Aspect 58. The composition of any one of aspects 46-57, further comprising at least one additional therapeutic agent.

Aspect 59. The composition of aspect 58, wherein the at least one additional therapeutic agent comprises a steroid.

Aspect 60. The composition of aspect 59, wherein the steroid comprises triamcinolone acetonide, betamethasone acetate, dexamethasone, or any combination thereof.

Aspect 61. The composition of any one of aspects 46-60, wherein the IGF inhibitor comprises chromeceptin and the anti-hypertensive agent comprises minoxidil.

Aspect 62. The composition of any one of aspects 46-60, wherein the IGF inhibitor comprises chromeceptin and the SERPINE1 serine protease inhibitor comprises tiplaxtinin.

Aspect 63. The composition of any one of aspects 46-60, wherein the IGF inhibitor comprises chromeceptin and the HDAC inhibitor comprises vorinostat.

Aspect 64. The composition of any one of aspects 46-60, wherein the anti-hypertensive agent comprises minoxidil and the SERPINE1 serine protease inhibitor comprises tiplaxtinin.

Aspect 65. The composition of any one of aspects 46-60, wherein the anti-hypertensive agent comprises minoxidil and verapamil.

Aspect 66. The composition of any one of aspects 46-60, wherein the one or more therapeutic agents comprise pirfenidone and nintedanib.

Aspect 67. The composition of any one of aspects 46-60, wherein the one or more therapeutic agents comprise verapamil and nintedanib.

Aspect 68. The composition of any one of aspects 46-60, wherein the one or more therapeutic agents comprise mitoxantrone and nintedanib.

Aspect 69. The composition of any one of aspects 46-60, wherein the one or more therapeutic agents comprise verapamil, mitoxantrone, and nintedanib.

Aspect 70. The composition of any one of aspects 46-60, wherein the one or more therapeutic agents comprise vorinostat, mitoxantrone, and nintedanib.

Aspect 71. The composition of any one of aspects 46-60, wherein the one or more therapeutic agents comprise minoxidil, mitoxantrone, and nintedanib.

Aspect 72. The composition of any one of aspects 46-60, wherein the one or more therapeutic agents comprise celecoxib and salinomycin.

Aspect 73. The composition of any one of aspects 46-60, wherein the one or more therapeutic agents comprise celecoxib and sulindac.

Aspect 74. The composition of any one of aspects 46-60, wherein the one or more therapeutic agents comprise salinomycin and sulindac.

Aspect 75. The composition of any one of aspects 46-60, wherein the one or more therapeutic agents comprise celecoxib, salinomycin, and sulindac.

Aspect 76. The composition of aspect 60, wherein the one or more therapeutic agents comprise nintedanib and the steroid comprises triamcinolone acetonide.

Aspect 77. The composition of aspect 60, wherein the one or more therapeutic agents comprise pirfenidone and nintedanib and the steroid comprises triamcinolone acetonide.

Aspect 78. The composition of aspect 60, wherein the one or more therapeutic agents comprise mitoxantrone and nintedanib and the steroid comprises triamcinolone acetonide.

Aspect 79. The composition of aspect 60, wherein the one or more therapeutic agents comprise verapamil and nintedanib and the steroid comprises triamcinolone acetonide.

Aspect 80. The composition of any one of aspects 46-79, wherein the composition comprises at least three of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that the composition does not comprise both a HDAC inhibitor and a HAT inhibitor.

Aspect 81. The composition of any one of aspects 46-80, wherein the composition comprises at least four of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that inhibits TGF-β signaling, a WNT pathway inhibitor, a lysyl oxidase-like 2 or lysyl oxidase-like 3 inhibitor, or any combination thereof, provided that the composition does not comprise both a HDAC inhibitor and a HAT inhibitor.

Aspect 82. The composition of any one of aspects 46-81, further comprising a pharmaceutically acceptable carrier or diluent.

Aspect 83. The composition of aspect 82, wherein the pharmaceutically acceptable carrier or diluent comprises petrolatum, cetyl alcohol, glyceryl monostearate, lanolin, stearic acid, methylcellulose, carboxymethylcellulose, stearyl alcohol, a carbomer, a polyethylene oxide, a polyoxyethylene-polyoxypropylene copolymer, polyvinylalcohol, hydroxypropyl cellulose, hydroxyethyl cellulose, hydroxypropyl methylcellulose, hydroxypropyl methylcellulose phthalate, gum tragacanth, xanthan gum, sodium alginate, gelatin, ethyl alcohol, glycerin, water, or any combination thereof.

Aspect 84. The composition of any one of aspects 46-83, wherein the composition is formulated as an ointment, lotion, cream, paste, gel, spray, or foam.

Aspect 85. A method for treating or delaying the onset, progression, or relapse of a fibrotic disorder in a subject, the method comprising administering to the subject the composition of any one of aspects 46-84.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the disclosure and are not intended to limit the scope of what the inventors regard as their disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1: Multiomics Approach to Identify Genes Driving Keloid Pathogenesis

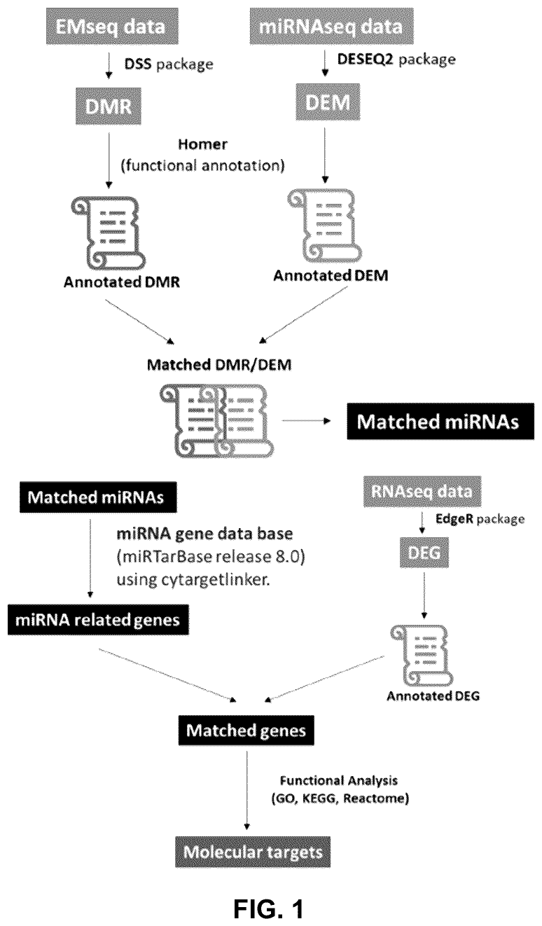

Herein, a multiplatform integrated genomics approach has been applied on the same set of patient derived keloid cells to identify multiple cellular pathways that can be targeted for keloid therapy using combinations of existing FDA approved or preclinical drugs applied topically. These drugs should greatly expand options for keloid therapy, especially through topical application, leading to a more personalized treatment options. It is possible that some of these drugs will also be effective in treating other fibrotic disorders that are driven by the same molecular pathways. Details of the workflow used for combining enzymatic DNA methylation sequencing (EMseq), microRNA sequencing (miRNAseq), and RNA sequencing (RNAseq) datasets are provided in FIG. 1. This workflow enabled a “multiomics” approach to identify the major genes driving keloid pathogenesis. EMseq performed in duplicate revealed 35,935 Differentially Methylated Regions (DMRs) in keloid DNA samples corresponding to 2,524 genes with enhanced DNA methylation and 4,546 genes with reduced DNA methylation. The EMseq data was correlated with the miRNAseq data on the targets of the Differentially Expressed miRNAs (DEMs) present in keloids, as well as the Differentially Expressed Genes (DEGs) identified by RNAseq. For example, since DNA methylation leads to gene silencing, a highly methylated gene from the EMseq dataset corresponding to its downregulated transcript levels in keloid samples in the RNAseq data, or vice versa, would be a positive correlation and generate more confidence in that candidate gene playing a potentially important role in keloid pathology. Similarly, a miRNA overexpressed in keloids with its target genes being downregulated in the RNAseq data, or vice versa, would also boost confidence that these genes are likely to be contributing to the keloid phenotype.

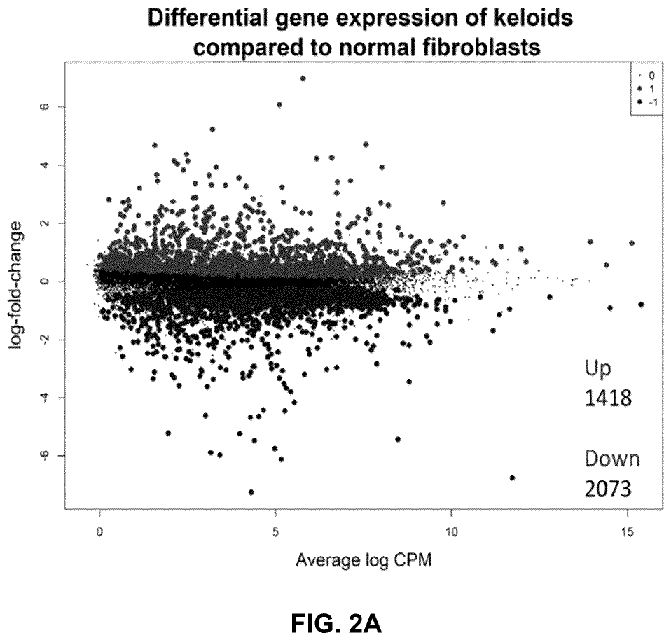

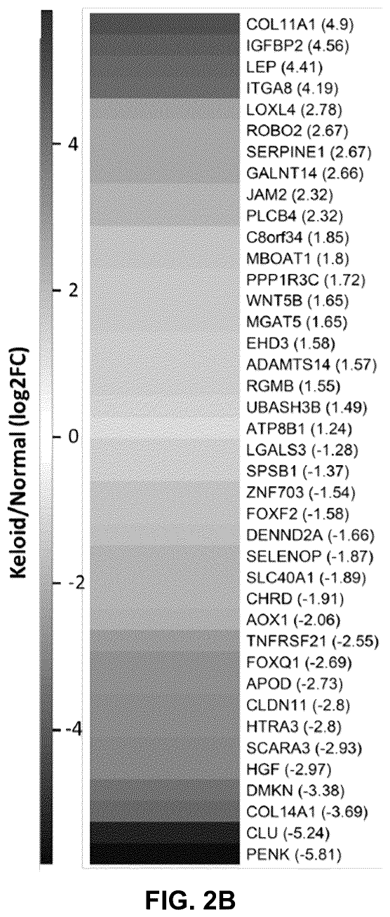

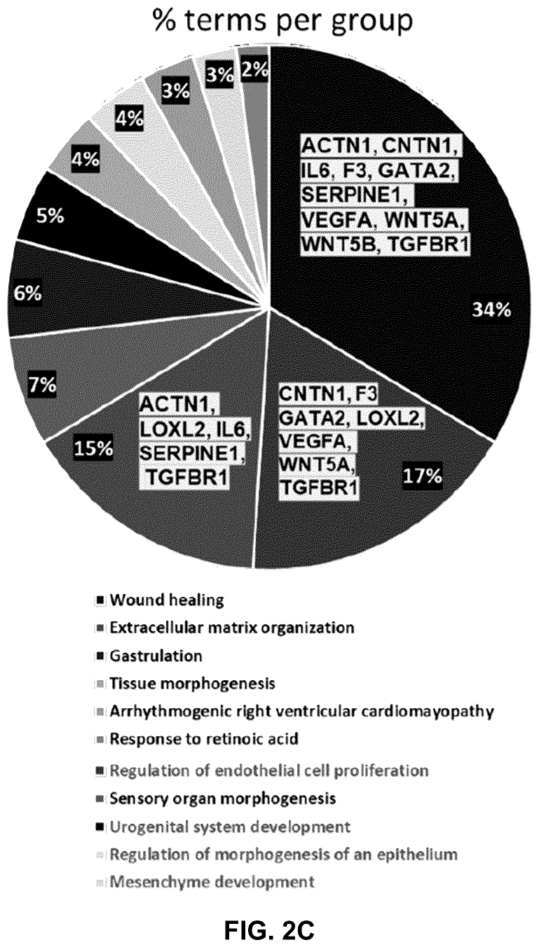

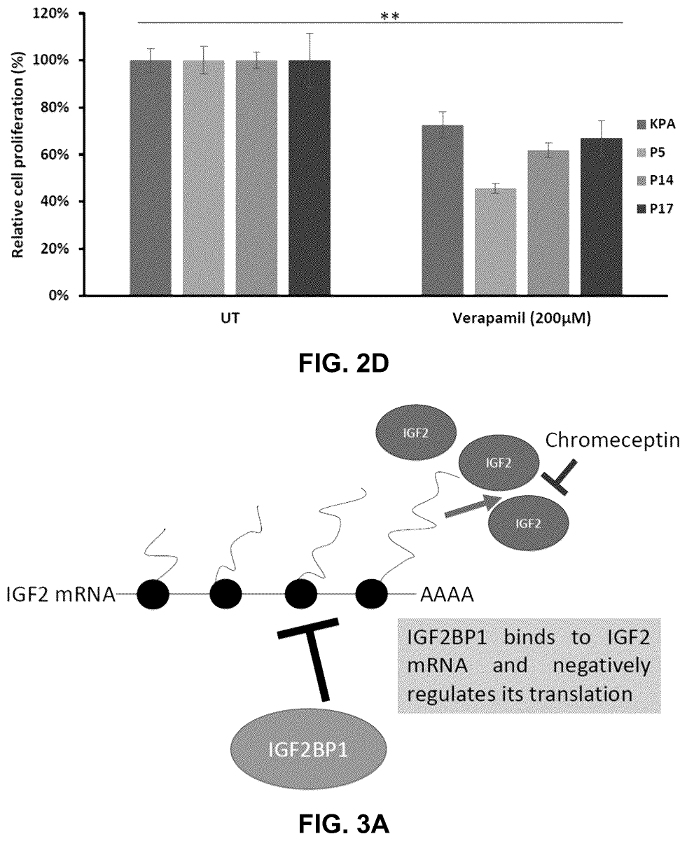

Identification of actionable genes and pathways driving keloid pathogenesis using our multiomics approach are shown in FIGS. 2A-2D. FIG. 2A: MA plot of RNA sequencing (RNAseq) performed in triplicate on 4 normal Human Dermal Fibroblasts (HDFs) and 6 keloid dermal fibroblast samples from patients (P) shows the upregulation of 1,418 and downregulation of 2,073 genes for a total of 3,491 Differentially Expressed Genes (DEGs) in keloid samples. Gray dots denote higher mean expression levels and black dots denote lower mean expression levels (log 2FC: log 2 fold change) in keloids dermal fibroblasts compared to the normal HDFs. FIG. 2B: Heatmap of the top 20 up- and downregulated genes identified in the RNAseq data are shown, several of which have been implicated in keloid pathogenesis previously, while others have not been studied so far in the context of keloids. FIG. 2C: Pie chart depicting the results of the integrated data from 3481 DEGs from RNAseq, 7070 DMRs from EMseq and the DEMs from miRNAseq data resulted in a relatively short list of 181 genes (132 genes that were upregulated 49 genes that were downregulated) that would be our high confidence candidate genes involved in keloid pathology. Consistent with this notion, two-thirds of these genes function in pathways that are involved in wound healing, extracellular matrix organization and regulation of endothelial cell proliferation—these pathways have been previously implicated in keloid pathogenesis as well as in other fibrotic diseases. Genes highlighted in yellow are known to contribute to fibrotic diseases or keloids via multiple pathways. This clearly validates our integrated multiomics approach for winnowing down large candidate gene lists from individual genomic assays to arrive at the most relevant genes contributing to the biological process under study. Several of the genes identified here have been evaluated as potential drug targets in the rest of figures shown here. Pathways highlighted in yellow potentially contribute to the regulation of blood pressure. High blood pressure, or hypertension, has been previously implicated in keloid pathology, although no molecular data in support of this idea was available previously. FIG. 2D: The FDA approved calcium channel blocking antihypertensive drug Verapamil inhibits the proliferation of keloid fibroblasts. Data from keloid dermal fibroblasts from 4 patients (KPA, P5, P14 and P17) are shown here.

### Example 2: Pathways Blocking Keloid Fibroblast Proliferation

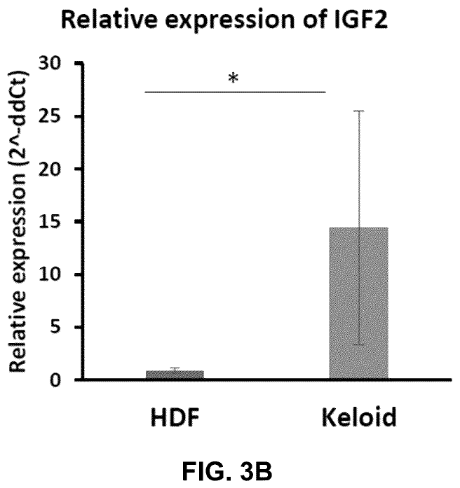

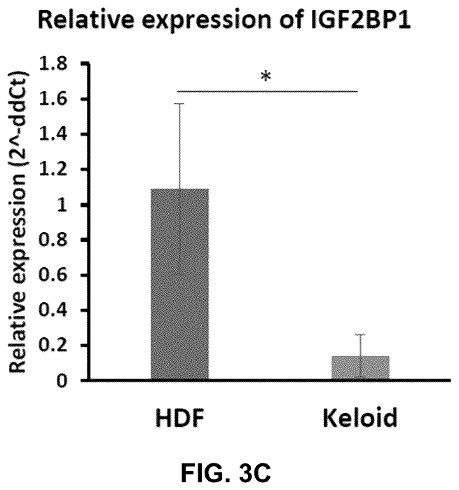

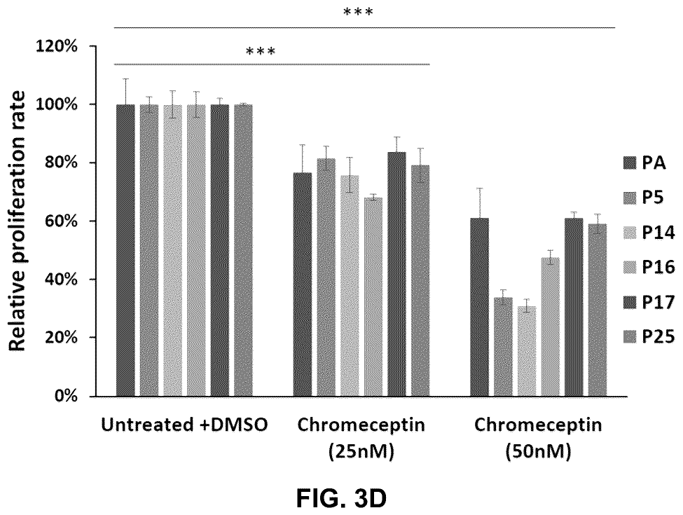

FIGS. 3A-3D show chromeceptin, an inhibitor of Insulin-like Growth Factor 2 (IGF2) signaling, blocks keloid fibroblast proliferation. FIG. 3A: Scheme depicting the negative translational regulation of IGF2 mRNA by the binding of Insulin-like Growth Factor 2 mRNA Binding Protein 1 (IGF2BP1). Chromeceptin is a pre-clinical inhibitor of IGF2 signaling that is effective in the low nanomolar range. The expression of IGF2 and IGF2BP1 are anti-correlated as shown by the data in next two figures. FIG. 3B: Quantitative RT-PCR based validation of the overexpression of IGF2, a signaling protein that stimulates growth and development, in keloid fibroblasts. Data was obtained from 3 normal primary human dermal fibroblasts (HDFs) and 3 keloid dermal fibroblasts. Relative expression was normalized to the levels of HPRT gene expression. Error bars represent standard deviation of the mean (mean±SD) obtained from three experiments. Significant differences are indicated by the asterisk (*P<0.05; ** P<0.005). FIG. 3C: Quantitative RT-PCR confirming the reduced expression of Insulin-like IGF2BP1, a negative regulator of IGF2 translation, in keloid fibroblasts. Experiment was performed as described above for FIG. 3B. FIG. 3D: Keloid dermal fibroblasts are sensitive to Chromeceptin. Data from keloid dermal fibroblasts from 6 patient (P) tissue samples numbered PA, P5, P14, P16, P17, and P25 are shown here.

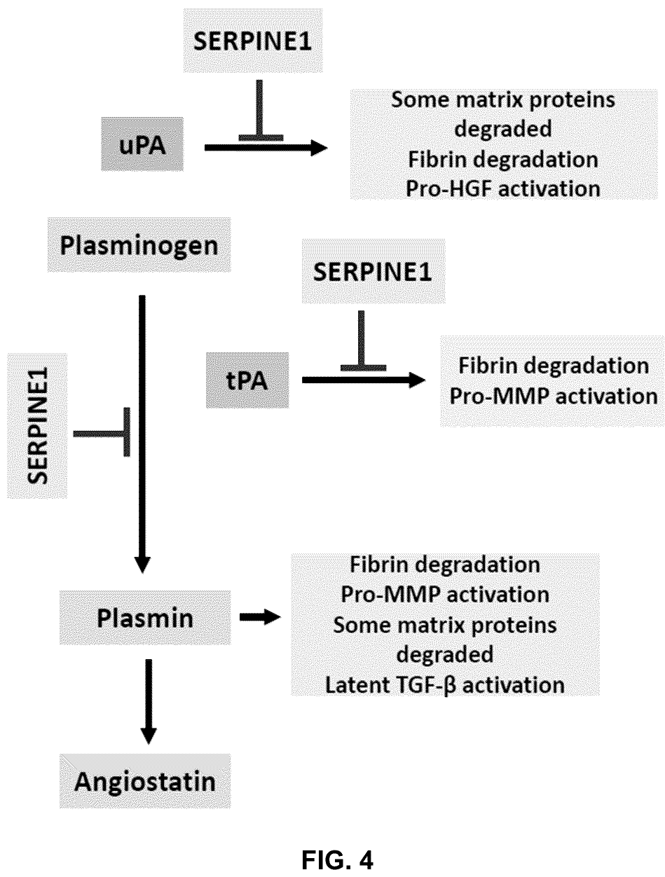

FIG. 4 shows physiological pathways regulated by SERPINE1 (Serine Proteinase Inhibitor, Family E, Member 1). SERPINE1, also known as Plasminogen Activator Inhibitor 1 (PAI1), is a protease inhibitor that regulates multiple pathways by blocking fibrin and extracellular matrix degradation, thereby leading higher levels of collagen deposition. Legend: urokinase-type plasminogen activator (uPA); tissue-type plasminogen activator (tPA); matrix metalloproteinase (MMP); hepatocyte growth factor (HGF); plasminogen activator inhibitor 1 (PAI1).

### Example 3: Effectively Blocking Keloid Fibroblast Proliferation Pathways

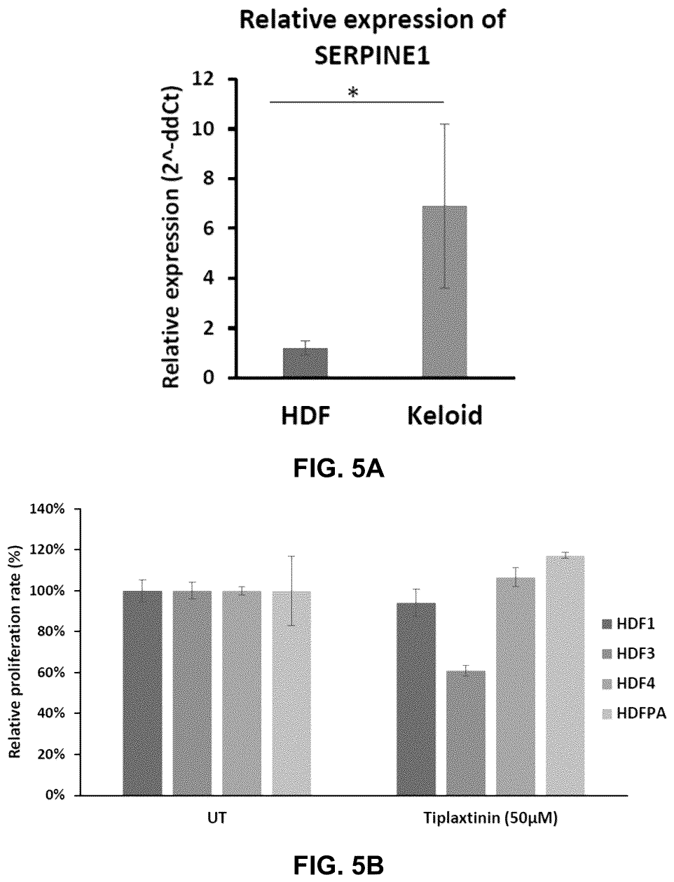

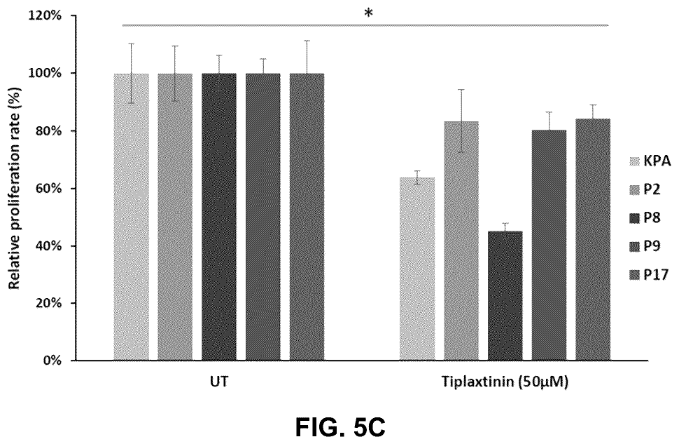

FIGS. 5A-5C show Tiplaxtinin, a SERPINE1 inhibitor, preferentially blocks keloid fibroblast proliferation over normal HDF proliferation. FIG. 5A: Quantitative RT-PCR to confirm the overexpression of SERPINE1 in keloid fibroblasts compared to HDFs. FIG. 5B: Normal primary HDFs are not significantly sensitive to Tiplaxtinin, a pre-clinical inhibitor of SERPINE1. FIG. 5C: Keloid dermal fibroblasts are sensitive to Tiplaxtinin in the low micromolar range.

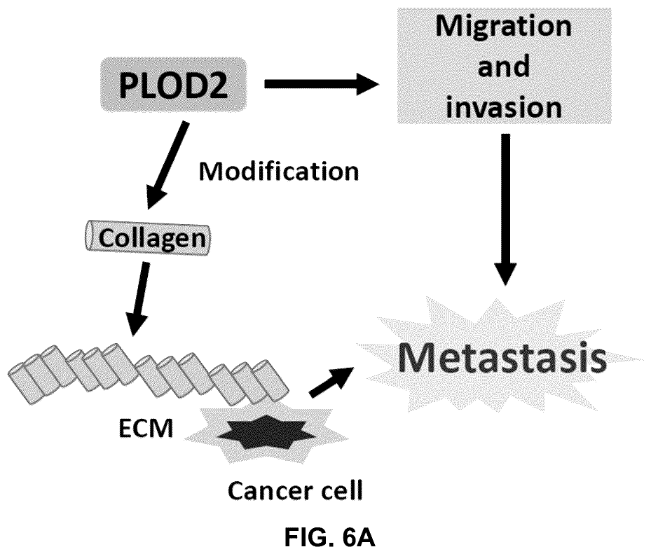

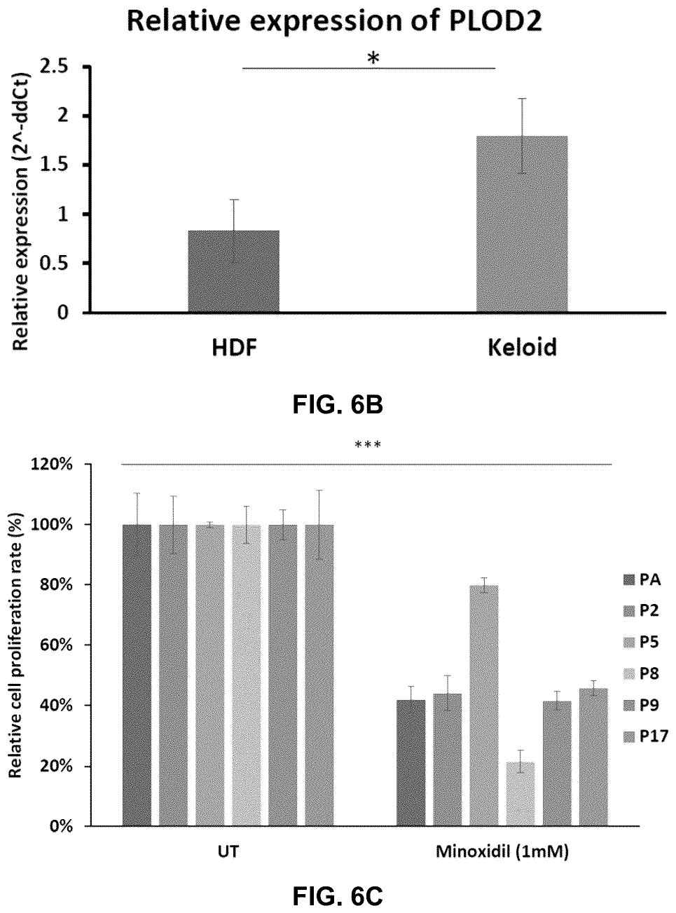

FIGS. 6A-6C show the FDA approved hair loss treatment Minoxidil inhibits Procollagen-Lysine, 2-Oxoglutarate 5-Dioxygenase 2 (PLOD2) and blocks keloid fibroblast proliferation FIG. 6A: Scheme showing the known roles of PLOD2 in regulating collagen maturation, thereby impacting the Extra Cellular Matrix (ECM), which in turn affects metastasis of cancer cells, and presumably keloid formation as well. PLOD2 catalyzes the hydroxylation of lysyl residues in collagen which is critical for the stability of intermolecular crosslinks in mature collagen. FIG. 6B: Quantitative RT-PCR to confirm the overexpression of PLOD2 in keloid fibroblasts compared to HDFs. FIG. 6C: Keloid dermal fibroblasts are sensitive to Minoxidil in the low-to-submillimolar range. Note: Minoxidil is FDA-approved for treating hair loss, where it is typically applied as a 5% (˜45 mM) topical solution.

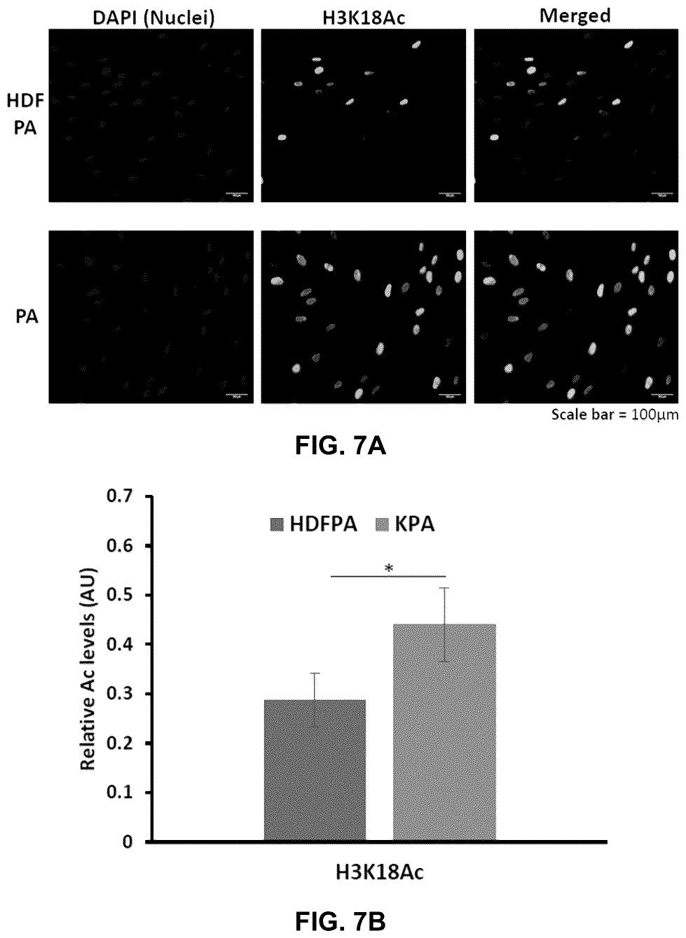

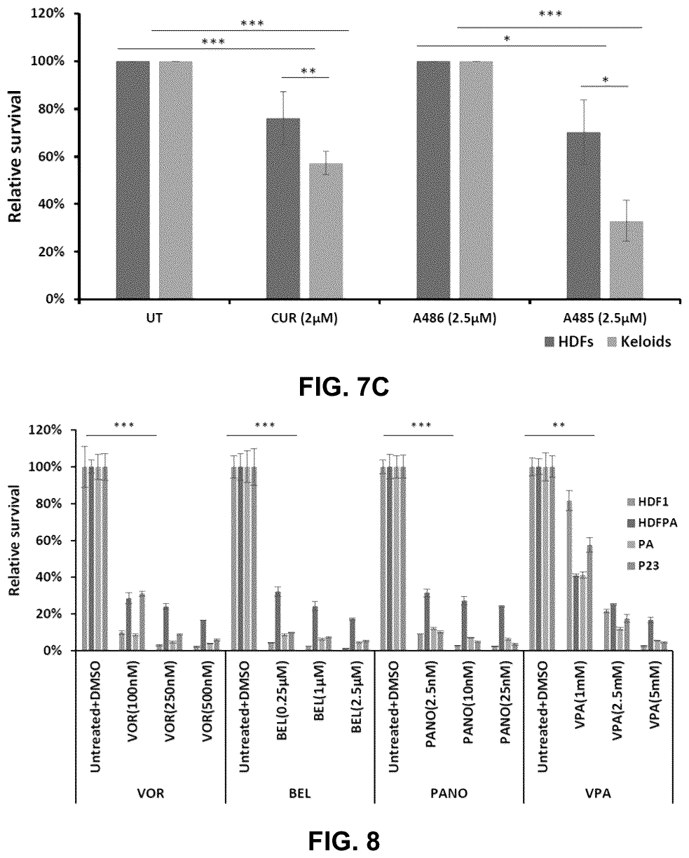

FIGS. 7A-7C show keloid fibroblasts exhibit higher levels of histone H3 lysine 18 acetylation (H3K18ac) and are sensitive to Histone Acetyltransferase (HAT) inhibitors, presumably due to their addition to higher levels of H3K18ac for survival and proliferation. FIG. 7A: Indirect immunofluorescence microscopy using H3K18ac specific antibodies reveals the presence of higher levels of this epigenetic mark in keloid fibroblasts (KPA) when compared to normal dermal fibroblasts (HDF PA) in the same patient. FIG. 7B: Quantitation of the data shown above in FIG. 7A. Ac=acetylation. FIG. 7C: Compared to normal HDFs, keloid fibroblasts are much more sensitive to both natural HAT inhibitors like Curcumin (CUR), as well as synthetic pre-clinical HAT inhibitor compounds such as A485. A structurally related dummy compound, A486, did not affect the proliferation of either HDFs or keloid fibroblasts and serves as a good negative control. Legend: histone acetyltransferase (HAT); curcumin (CUR; a natural HAT inhibitor): dummy compound (A486); synthetic HAT inhibitor (A485).

FIG. 8 shows keloid fibroblasts are exquisitely sensitive to multiple FDA approved Histone Deacetylase (HDAC) inhibitors, presumably due to their addition to higher levels of H3K18ac for survival and proliferation. Since keloid fibroblasts exhibit higher levels of H3K18ac (FIGS. 7A-7B), it is possible that treatment with HDAC inhibitors may enhance their acetylation levels even further leading to cytotoxicity. We tested this possibility and found that both keloid fibroblasts and normal HDFs show substantial sensitivity to FDA approved HDAC inhibitors. However, since keloids are a dermatological disorder, it would still be possible to apply these drugs topically or deliver them via intralesional injections in a manner that is localized just to the keloids to derive therapeutic benefits. Legend: Ac (acetylation); vorinostat (VOR); belinostat (BEL); panobinostat (PANO); valproic acid (VPA).

### Example 4: Combinatorial Therapy to Combat Keloid Fibroblast Proliferation

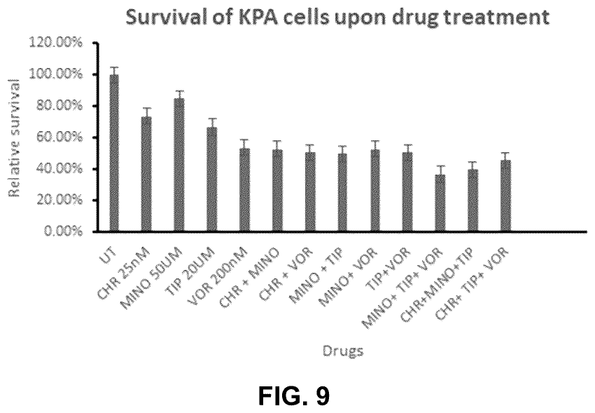

FIG. 9 shows synergistic effects of combination drug treatment on keloid fibroblast proliferation using FDA approved and pre-clinical drugs. Combination drug treatment targeting keloid fibroblast proliferation based on the actionable genes and pathways identified through our multiomics approach or basic research, can lead to synergistic inhibition of keloid fibroblast proliferation at lower doses of individual drugs. A proof of principle experiment is shown demonstrating the enhanced efficacy of a combination of two drugs in inhibiting keloid fibroblast proliferation compared to the individual drugs. In principle, multiple drugs targeting different pathways that independently contribute to keloid formation can be used simultaneously to achieve synergistic benefits in keloids therapy, especially when applied topically to avoid systemic effects. Legend: chromoceptin (CHR); minoxidil (MINO); tiplaxtinin (TIP); vorinostat (VOR).

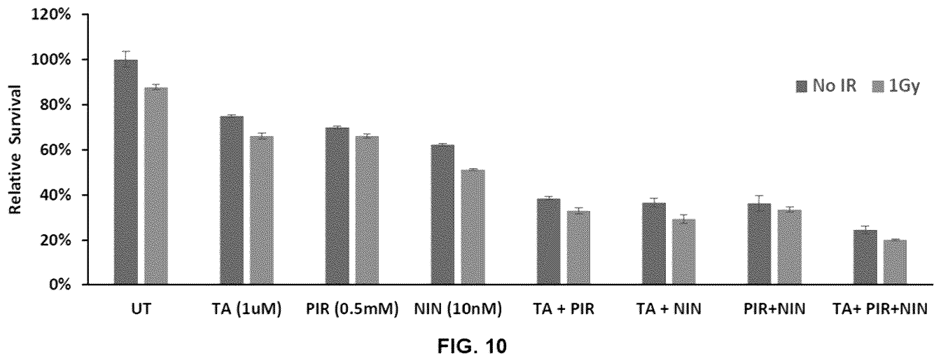

FIG. 10 shows proliferation of keloid fibroblasts is blocked in a synergistic manner upon treatment with a combination of FDA approved anti-inflammatory and antifibrotic drugs. Patient derived KPA keloid fibroblasts were either left untreated or treated with the indicated concentrations of the drugs, either with or without a low 1 Gy dose of radiation, and surviving cells were counted one week later. Error bars represent standard deviation. TA=Triamcinolone Acetonide, and FDA approved inti-inflammatory steroid commonly used in keloid therapy; PIR=Pirfenidone, a FDA approved antifibrotic drug that works by blocking TGF-β activities that drive keloid disease; NIN=Nintedanib, a FDA approved antifibrotic kinase inhibitor that blocks the function of several growth factors important for keloid fibroblast proliferation; IR=Ionizing Radiation, which has been previously shown by us to be effective in blocking keloid fibroblast proliferation in vitro and preventing keloid recurrence in patients following their surgical excision. Note that this assay cannot accurately determine cell survival below ˜20% due to the presence of significant numbers of senescent cells that are still alive, but which are not capable of proliferating or dividing any more. The use of a higher dose of radiation is likely to result in further synergy in the anti-proliferative effects of the drugs used here. The synergistic effects of the drugs are likely due to the fact that they have different targets in the pro-fibrotic pathways and suggest that significantly improved therapeutic benefits can be obtained by combining these drugs rather than using them individually for keloid therapy.

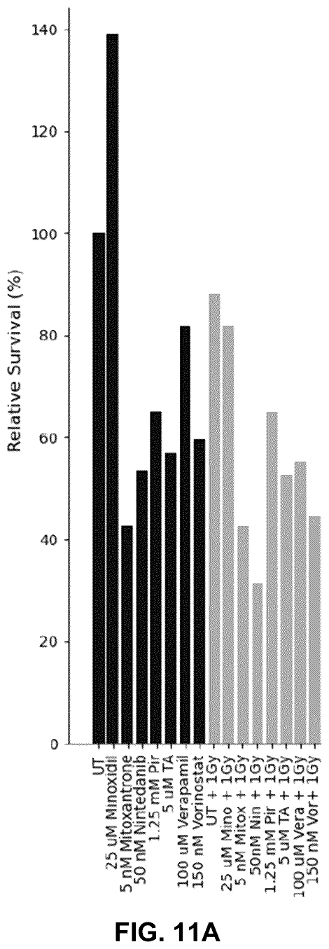

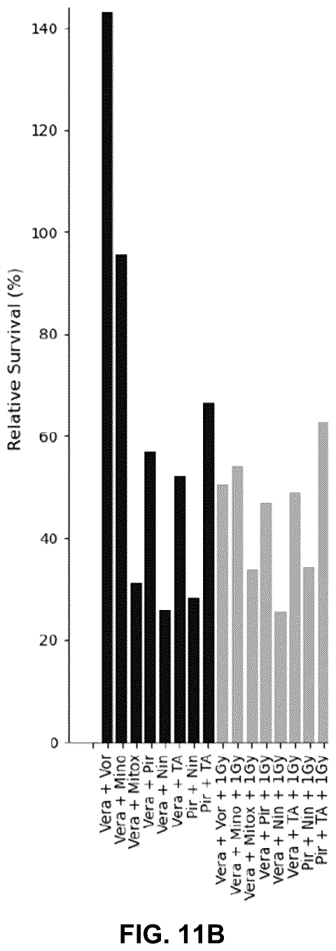

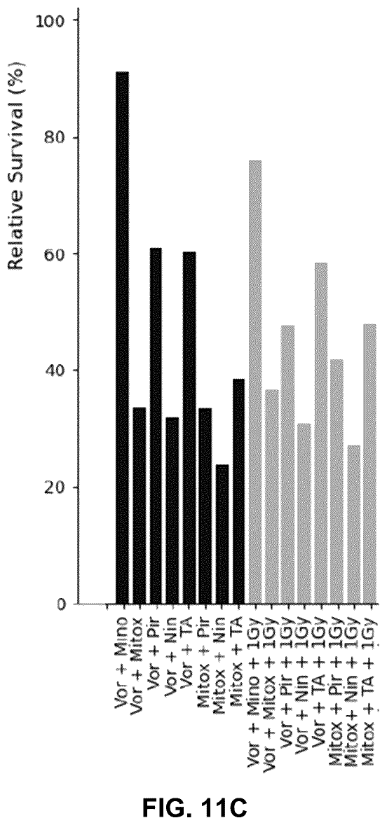

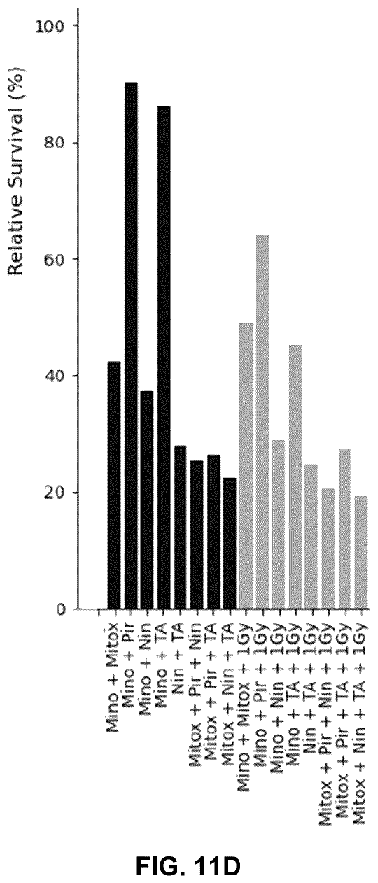

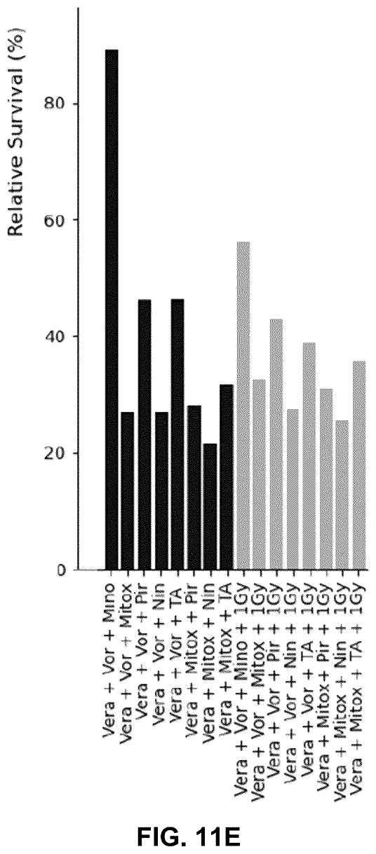

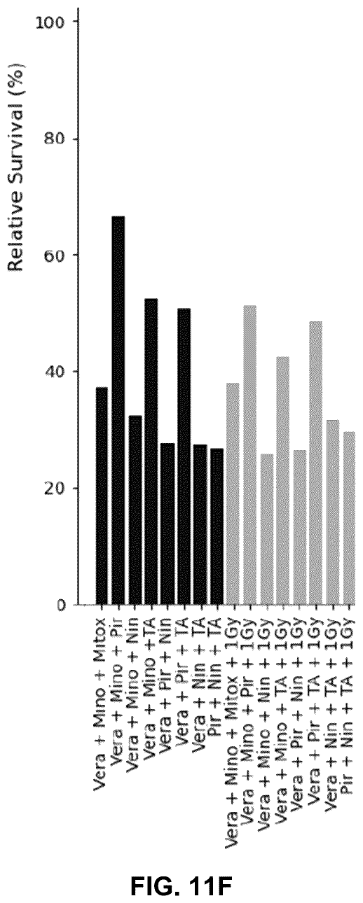

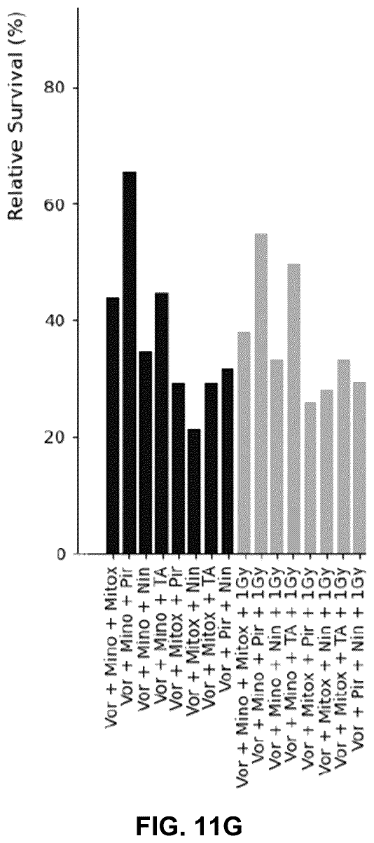

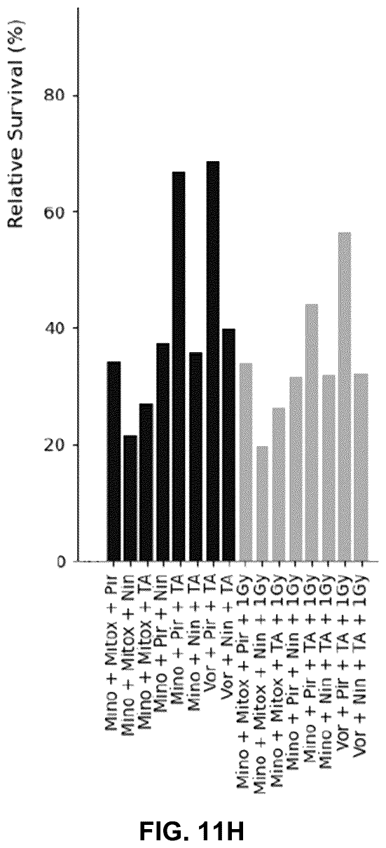

FIGS. 11A-11H show proliferation of keloid fibroblasts is blocked in a synergistic manner upon treatment with multiple combinations of FDA approved drugs along with radiation. The experiment was performed as described in FIG. 10 using the indicated concentrations of drugs, either without radiation (dark bars) or with ionizing radiation (gray bars). Drugs used here are: Mino=Minoxidil, an inhibitor of Procollagen-Lysine, 2-Oxoglutarate 5-Dioxygenase 2 (PLOD2); Mitox=Mitoxantrone, a DNA intercalating Topoisomerase II inhibiting antineoplastic chemotherapeutic; Nin=Nintedanib, an FDA approved antifibrotic kinase inhibitor; Pir=Pirfenidone, an FDA approved antifibrotic drug; TA=Triamcinolone Acetonide, an FDA approved anti-inflammatory steroid; Vera=Verapamil, a calcium channel inhibiting antihypertensive drug; Vor=Vorinostat, a histone deacetylase (HDAC) inhibitor.

### Example 5: Additional Genes Upregulated in Keloid Fibroblasts

FIGS. 12A-12B show the WNT pathway is upregulated in keloid fibroblasts that are sensitive to inhibition of the WNT pathway using repurposed FDA approved drugs. FIG. 12A shows qRT-PCR based analysis to confirm the differential mRNA expression of WNT5A in keloid fibroblasts relative to normal human dermal fibroblasts (HDF). Primary keloid fibroblasts were obtained and processed for qRT-PCR 3 normal fibroblasts and 3 keloid fibroblasts from 6 patients. Relative expression was normalized to the levels of HPRT gene expression. Error bars represent standard deviation of the mean obtained from three experiments. FIG. 12B shows patient derived keloid fibroblasts were either left untreated or treated with the indicated low concentrations of the FDA approved drugs that are also known to inhibit WNT signaling. Higher concentrations of the WNT inhibiting drugs are expected to have stronger effects. CEL=Celecoxib; Salinomycin=SLM; SUL=Sulindac.

FIG. 13 shows lysyl oxidase-like 2 (LOXL2) gene expression is upregulated in keloid fibroblasts. qRT-PCR based analysis to confirm the differential mRNA expression of LOXL2 in keloid fibroblasts relative to normal human dermal fibroblasts (HDF). Primary keloid fibroblasts were obtained and processed for qRT-PCR 3 normal fibroblasts and 3 keloid fibroblasts from 6 patients. Relative expression was normalized to the levels of HPRT gene expression. Error bars represent standard deviation of the mean obtained from three experiments. Significant differences are indicated by the asterisk (*P<0.05).

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

What is claimed is:

1. A method for treating or delaying the onset, progression, or relapse of a fibrotic disorder in a subject, the method comprising administering to the subject one or more therapeutic agents comprising a SERPINE1 serine protease inhibitor, a histone acetyltransferase (HAT) inhibitor, a histone deacetylase (HDAC) inhibitor, an insulin-like growth factor (IGF) inhibitor, an anti-hypertensive agent, a topoisomerase II inhibitor, a tyrosine kinase inhibitor, an agent that downregulates growth factors, or any combination thereof, provided that a HAT inhibitor and a HDAC inhibitor are not simultaneously administered to the subject.

2. The method of claim 1, wherein the fibrotic disorder comprises keloid, pulmonary fibrosis, hepatic fibrosis, cardiac fibrosis, renal fibrosis, mediastinal fibrosis, retroperitoneal cavity fibrosis, bone marrow fibrosis, scleroderma, or any combination thereof.

3. The method of claim 1, wherein the one or more therapeutic agents are administered topically, parenterally, intralesionally, orally, or any combination thereof.

4. The method of claim 1, wherein the SERPINE1 serine protease inhibitor comprises tiplaxtinin, annonacinone, aleplasinin, diaplasinin, CDE-096, AZ3976, TM5275, TM5007, TM5441, ACT001, toddalolactone, loureirin B, embelin, or geodin, antibodies or nanobodies targeting SERPINE1, or any combination thereof.

5. The method of claim 1, wherein the HAT inhibitor is natural or synthetic, and wherein the HAT inhibitor comprises curcumin, garcinol, anacardic acid, C646, CPTH2, A485, or any combination thereof.

6. The method of claim 1, wherein the HDAC inhibitor comprises valproic acid, sodium valproate, Panobinostat, Belinostat, Vorinostat, Romidepsin, or any combination thereof.

7. The method of claim 1, wherein the IGF inhibitor comprises chromeceptin Linsitinib, Ceritinib, ginsenoside, picropodophyllin, or any combination thereof.

8. The method of claim 1, wherein the anti-hypertensive agent comprises verapamil, minoxidil, hydralazine, nitroglycerin, amlodipine, diltiazem, losartan, telmisartan, captopril, lisinopril, atenolol, propranolol, prazosin, hydrochlorothiazide, furosemide, spironolactone, or any combination thereof.

9. The method of claim 1, wherein the topoisomerase II inhibitor comprises mitoxantrone.

10. The method of claim 1, wherein the tyrosine kinase inhibitor comprises nintedanib.

11. The method of claim 1, wherein the agent that downregulates growth factors comprises pirfenidone.

12. The method of claim 1, wherein the fibrotic disorder comprises keloid and wherein the method further comprises administering superficial radiation therapy to the subject.

13. The method of claim 1, further comprising performing surgical resection.

14. The method of claim 1, further comprising administering a steroid to the subject, wherein the steroid comprises triamcinolone acetonide, betamethasone acetate, dexamethasone, or any combination thereof.

15. The method of claim 1, wherein the method comprises administering at least three of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that downregulates growth factors, or any combination thereof, provided that the composition does not comprise both a HDAC inhibitor and a HAT inhibitor.

16. The method of claim 15, wherein the one or more therapeutic agents comprise minoxidil, tiplaxtinin, and vorinostat.

17. The method of claim 15, wherein the one or more therapeutic agents comprise chromeceptin, minoxidil, and tiplaxtinin.

18. The method of claim 15, wherein the one or more therapeutic agents comprise vorinostat, mitoxantrone, and nintedanib.

19. The method of claim 15, wherein the one or more therapeutic agents comprise minoxidil, mitoxantrone, and nintedanib.

20. The method of claim 1, wherein the method comprises administering at least four of a SERPINE1 serine protease inhibitor, a histone acetyltransferase (HAT) inhibitor, a histone deacetylase (HDAC) inhibitor, an insulin-like growth factor (IGF) inhibitor, an antihypertensive agent, a topoisomerase II inhibitor, a tyrosine kinase inhibitor, an agent that downregulates growth factors, or any combination thereof, provided that the composition does not comprise both a HDAC inhibitor and a HAT inhibitor.
